(19) United States
(12) Patent Application Publication
(10) Pub. No.: US 2009/0104265 A1
(43) Pub. Date: Apr. 23, 2009

(54) Polymorphs of N-(4-chloro-3-methyl-5-isoxazolyl) 2-[2-methyl-4,5-(methylenedioxy)phenylacetyl] thiophene-3-sulfonamide, sodium salt

(76) Inventors: John Reichwein, Houston, TX (US); Timothy Hanser, New London, CT (US)

Correspondence Address: JONES DAY, 222 EAST 41ST ST, NEW YORK, NY, 10017, US

(21) Appl. No.: 11/717,498

(22) Filed: Mar. 12, 2007

Related U.S. Application Data
(60) Provisional application No. 60/781,861, filed on Mar. 13, 2006.

(51) Int. Cl. A61K 9/28; C07D 409/14; A61K 31/422; C07K 14/705

(52) U.S. Cl. 424/474; 548/246; 514/380; 530/408

(57) ABSTRACT

N-(4-chloro-3-methyl-5-isoxazolyl)-2-[2-methyl-4,5-(methylenedioxy)phenyl-acetyl]thiophene-3-sulfonamide, sodium salt, is provided herein in the form of three polymorphs (Forms A, B and C). Forms A, B and C are specified by the peaks in their X-ray powder diffraction patterns, their absorption peaks in their infrared absorption spectra, their peaks in their Raman spectra and their melting points.

This application claims priority to U.S. provisional application Ser. No. 60/781,861 filed march 13, 2006, entitled “POLYMORPHS OF N-(4-CHLORO-3-METHYL-5-ISOXAZOLYL) 2-[2-METHYL-4,5 METHYLENEDIOXY) PHENYLACETYL] THIOPHENE-3-SULFONAMIDE, SODIUM SALT” to Reichwein et al. The disclosure of the above referenced application is incorporated by reference herein.

### FIELD OF THE INVENTION

Provided herein are polymorphs of N-(4-chloro-3-methyl-5-isoxazolyl)-2-[2-methyl-4,5-(methylenedioxy)phenylacetyl]thiophene-3-sulfonamide, sodium salt, and processes for producing them.

### BACKGROUND

N-(4-chloro-3-methyl-5-isoxazolyl)-2-[2-methyl-4,5-(methylenedioxy)phenyl-acetyl]-thiophene-3-sulfonamide, sodium salt, modulates the activity of the endothelin family of peptides and is useful for the treatment of endothelin-mediated disorders. Due to the nature of these disorders, this compound's use as a pharmaceutical product may require storage for an extended period of time. Thus, the stability of this compound (bulk pharmaceutical chemicals) against heat and humidity during the storage period is very important. Therefore, a more stable form of this compound is desired.

### SUMMARY

It has been found that polymorphs of N-(4-chloro-3-methyl-5-isoxazolyl)-2-[2-methyl-4,5-(methylenedioxy)phenylacetyl]thiophene-3-sulfonamide, sodium salt, Forms A and B; a methyl t-butyl ether solvate, Form C; and an amorphous form, can be selectively produced on an industrial scale by crystallization from appropriate solvents and conditions. Further, Form B and mixtures of Forms A and B, can be interconverted to the more stable Form A under suitable conditions.

The amorphous form of sitaxsentan sodium is highly hygroscopic whereas the crystalline form is not (amorphous gains 22% of its total weight at 95% RH; crystalline gains less than 1.5% of their weight at 95% RH). Interconversion studies found polymorph A to be the more thermodynamically stable form. Without being bound to any theory, it is believed that the amorphous state converts with time to a mixture of polymorphs.

In particular, polymorphs of N-(4-chloro-3-methyl-5-isoxazolyl)-2-[2-methyl-4,5-(methylenedioxy)phenylacetyl]thiophene-3-sulfonamide, sodium salt, Forms A and B, having the chemical structure:

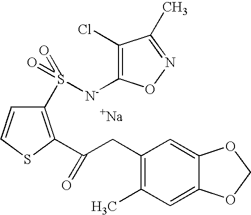

can be selectively produced and are distinguishable based upon their characteristic peaks in their X-ray powder diffraction (XRPD) patterns, infrared absorption spectra, Raman spectra and melting points.

Methods and Conditions for the Measurement of XRPD Patterns Method of the Measurement

The XRPD analysis was conducted on a Shimadzu XRD-6000-X-ray powder diffractometer on the samples by the following conditions.

Condition of the Measurement

| | |
|---|---|
| Target | Cu Ka |
| Filter | monochro |
| Voltage | 40 kV |
| Current | 40 mA |
| Slit IDS | RS 0.15 mm SS 1° |
| Scan speed | 3°/min |
| Range | 2.5 to 40 |

Method and Condition for the Measurement of Infrared Absorption

The thermalgravimetric infrared (TG/IR) absorption spectra were acquired on a TA Instrument TGA 2050 interfaced with a Nicolet model 560 Fourier transform infrared (FT-IR) spectrophotometer.

Method and Condition for the Measurement of Raman Absorption

The Raman spectra were acquired on a Raman bench interfaced to a Nicolet Magna 860 FT-IR spectrophotometer.

### Polymorph A (Form A)

The major peaks in the XRPD pattern of Form A expressed in degrees 2-theta are at approximately 6.72, 15.96, 22.38, 23.38 and 26.22.

FIGS. 1-8 show the XRPD pattern of Form A.

The major peaks (cm⁻¹) in the Raman spectra of Form A are at approximately 1697.4, 1602.1, 1489.8 and 1402.2 cm⁻¹.

FIG. 9 shows the Raman spectra of Form A.

Based on the characterization data, Form A appears to be a crystalline, nonhygroscopic solid which decomposes at approximately 200° C.

### Polymorph B (Form B)

The major peaks in the XRPD pattern of Form B expressed in degrees 2-theta are at approximately 6.6, 15.52, 18.38, 18.94 and 22.72.

FIG. 1 shows the XRPD pattern of Form B.

The major peaks (cm⁻¹) in the Raman spectra of Form B are at approximately 1696.9, 1594.7, 1490.2 and 1397.8 cm⁻¹.

FIG. 22 shows the Raman spectra of Form B.

Based on the characterization data, Form B appears to be a nonsolvated, crystalline material which decomposes around 203° C.

### Polymorph C (Form C)

The major peaks in the XRPD pattern of Form C expressed in degrees 2-theta are at approximately 5.14, 23.48 and 26.78.

FIG. 1 shows the XRPD pattern of Form C.

FIG. 23 shows the infrared absorption spectra of Form C.

Based on the characterization data, Form C appears to be a methyl t-butyl ether solvate of the compound.

### BRIEF DESCRIPTION OF THE DRAWINGS

FIG. 1 is the XRPD patterns of the polymorphs A, B, C and amorphous form.

FIG. 2 is the XRPD pattern of the polymorph A, sample lot I.

FIG. 3 is the XRPD pattern of the polymorph A, sample lot II.

FIG. 4 is the XRPD pattern of the polymorph A, sample lot III.

FIG. 5 is the XRPD pattern of the polymorph A, sample lot IV.

FIG. 6 is the XRPD pattern of the polymorph A, sample lot V.

FIG. 7 is the XRPD pattern of the polymorph A, sample lot VI.

FIG. 8 is the XRPD pattern of the polymorph A, sample lot VII.

FIG. 9 is the Raman absorption spectra of the polymorph A.

FIG. 10 is the DSC of the polymorph A, sample lot I.

FIG. 11 is the DSC of the polymorph A, sample lot IV.

FIG. 12 is the DSC of the polymorph A, sample lot III.

FIG. 13 is the TG of the polymorph A, sample lot I.

FIG. 14 is the TG of the polymorph A, sample lot IV.

FIG. 15 is the TG of the polymorph A, sample lot III.

FIG. 16 is the moisture sorption/desorption of the polymorph A, sample lot I.

FIG. 17 is the moisture sorption/desorption of the polymorph A, sample lot IV.

FIG. 18 is the moisture sorption/desorption of the polymorph A, sample lot III.

FIG. 19 is the DSC of the polymorph B.

FIG. 20 is the TG of the polymorph B.

FIG. 21 is the moisture sorption/desorption of the polymorph B.

FIG. 22 is the Raman absorption spectra of the polymorph B.

FIG. 23 is the TG/IR absorption spectra of the polymorph C.

FIG. 24 is the TG of the polymorph C.

FIG. 25 is the TG/IR absorption spectra of the polymorph A.

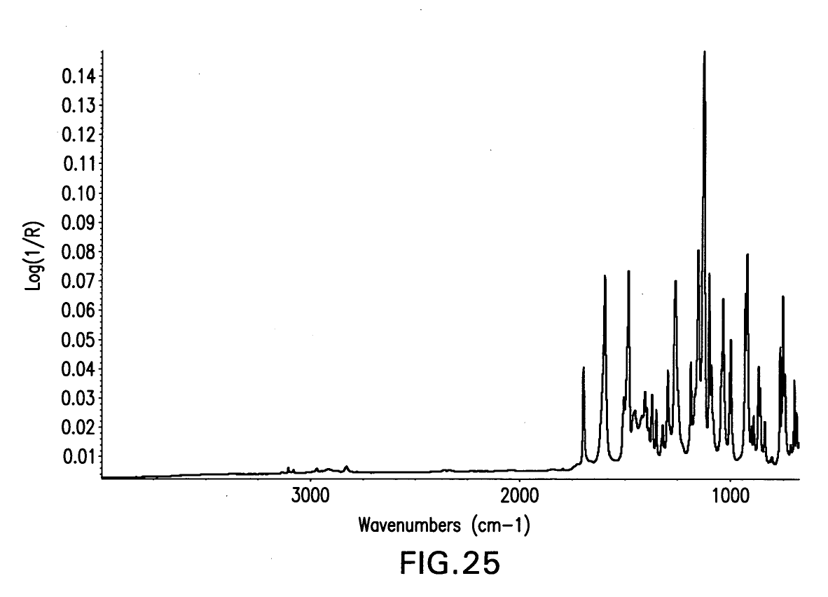

FIG. 26 is the TG/IR absorption spectra of the polymorph B.

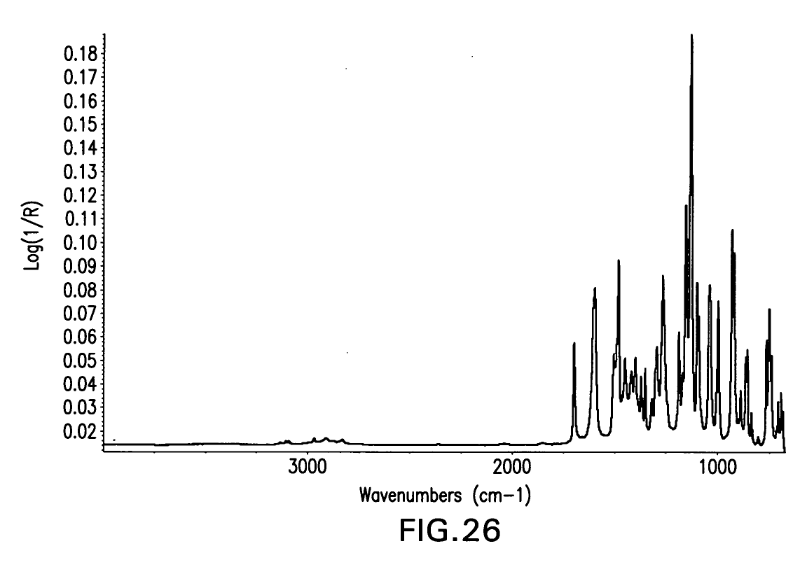

FIG. 27 is the XRPD pattern of the polymorph B, sample lot I.

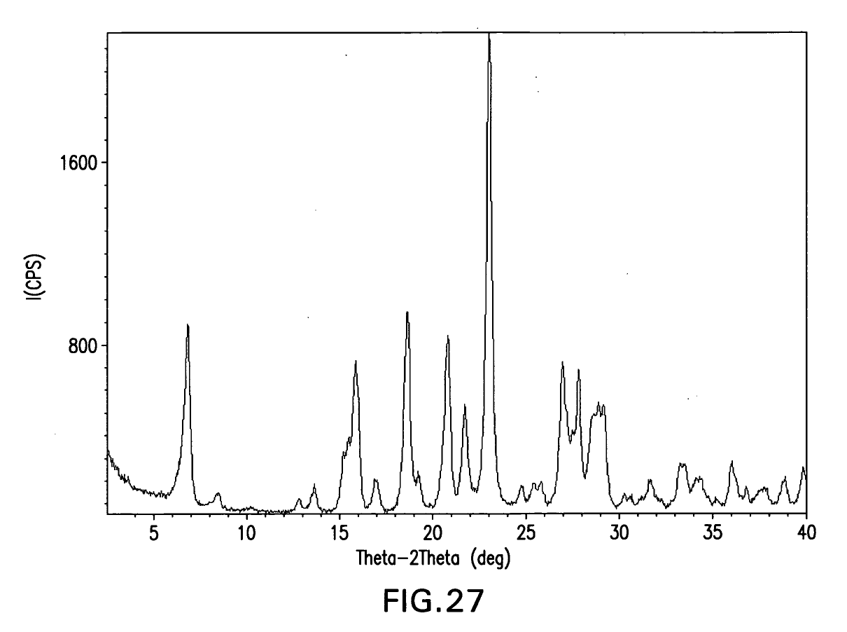

FIG. 28 is the XRPD pattern of the polymorph B, sample lot II.

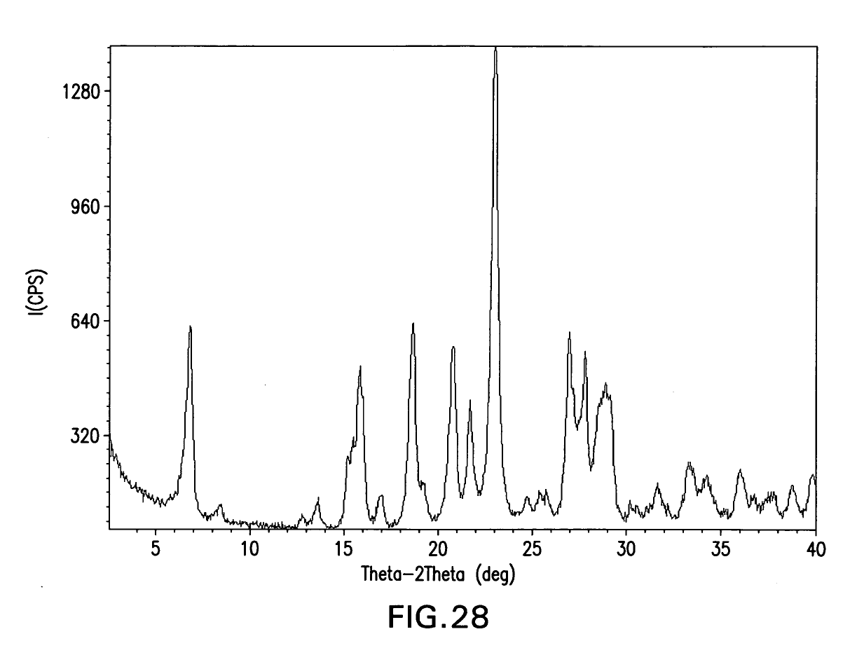

FIG. 29 is the XRPD pattern of the polymorph B, sample lot III.

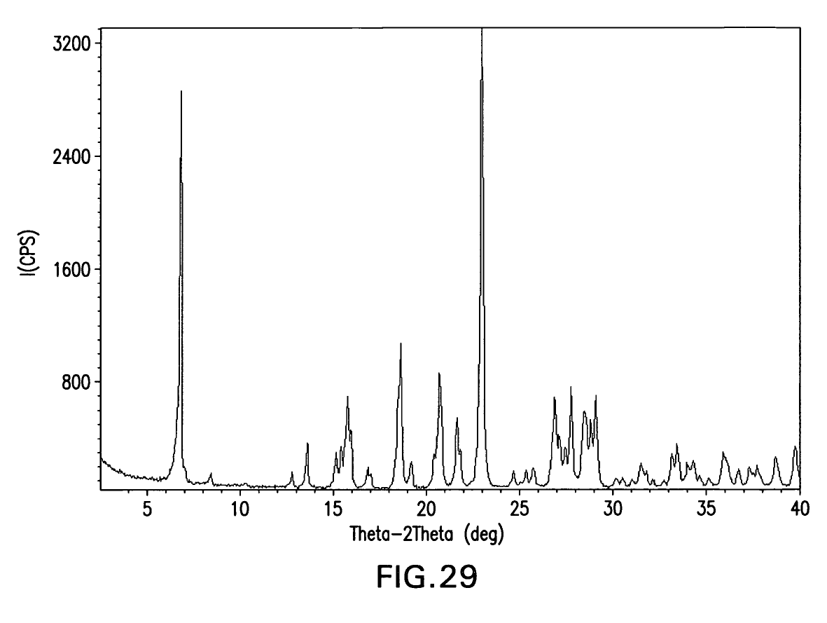

### DETAILED DESCRIPTION

### A. Definitions

Unless defined otherwise, all technical and scientific terms used herein have the same meaning as is commonly understood by one of skill in the art to which this subject matter belongs. All patents and publications referred to herein are incorporated by reference.

As used herein “sitaxsentan sodium” refers to N-(4-chloro-3-methyl-5-isoxazolyl)-2-[2-methyl-4,5-(methylenedioxy)phenylacetyl]-thiophene-3-sulfonamide, sodium. Other chemical names for sitaxsentan sodium include 4-chloro-3-methyl-5-(2-(2-(6-methylbenzo[d][1,3]dioxol-5-yl)acetyl)-3-thienylsulfonamido)isoxazole, sodium and N-(4-chloro-3-methyl-5-isoxazolyl)-2-[3,4-(methylenedioxy)-6-methylphenylacetyl]-thiophene-3-sulfonamide, sodium. The chemical structure of sitaxsentan sodium salt is described elsewhere herein.

As used herein, endothelin (ET) peptides include peptides that have substantially the amino acid sequence of endothelin-1, endothelin-2 or endothelin-3 and that act as potent endogenous vasoconstrictor peptides.

As used herein, an endothelin-mediated condition is a condition that is caused by abnormal endothelin activity or one in which compounds that inhibit endothelin activity have therapeutic, use. Such diseases include, but are not limited to hypertension, cardiovascular disease, asthma, inflammatory diseases, opthalmologic disease, menstrual disorders, obstetric conditions, gastroenteric disease, renal failure, pulmonary hypertension, interstitial lung disease, diastolic heart failure, endotoxin shock, anaphylactic shock, or hemorrhagic shock. Endothelin-mediated conditions also include conditions that result from therapy with agents, such as erythropoietin and immunosuppressants, which elevate endothelin levels.

As used herein an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease. In another embodiment, the amount is administered in order to ameliorate one or more symptoms of the disease. In other embodiments, repeated administration is required to achieve the desired amelioration of symptoms.

As used herein, an endothelin agonist is a compound that potentiates or exhibits a biological activity associated with or possessed by an endothelin peptide.

As used herein, an endothelin antagonist is a compound, such as a drug or an antibody, that inhibits endothelin-stimulated vasoconstriction and contraction and other endothelin-mediated physiological responses. The antagonist may act by interfering with the interaction of the endothelin with an endothelin-specific receptor or by interfering with the physiological response to or bioactivity of an endothelin isopeptide, such as vasoconstriction. Thus, as used herein, an endothelin antagonist interferes with endothelin-stimulated vasoconstriction or other response or interferes with the interaction of an endothelin with an endothelin-specific receptor, such as ET_{A} receptors, as assessed by assays known to those of skill in the art.

The effectiveness of potential agonists and antagonists can be assessed using methods known to those of skill in the art. For example, endothelin agonist activity can be identified by its ability to stimulate vasoconstriction of isolated rat thoracic aorta or portal vein ring segments (Borges et al. (1989) “Tissue selectivity of endothelin” Eur. J. Pharmacol. 165: 223-230).

As used herein a sulfonamide that is ET_{A} selective refers to sulfonamides that exhibit an IC₅₀ that is at least about 10-fold lower with respect to ET_{A} receptors than ET_{B} receptors.

As used herein, a sulfonamide that is ET_{B}, selective refers to sulfonamides that exhibit an IC₅₀ that is at least about 10-fold lower with respect to ET_{B}, receptors than ET_{A} receptors.

As used herein, treatment means any manner in which the symptoms of a conditions, disorder or disease are ameliorated or otherwise beneficially altered. Treatment also encompasses any pharmaceutical use of the compositions herein, such as use as contraceptive agents.

As used herein, amelioration of the symptoms of a particular disorder by administration of a particular pharmaceutical composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures.

As used herein, increased stability of a formulation means that the percent of active component present in the formulation, as determined by assays known to those of skill in the art, such as high performance liquid chromatography, gas chromatography and the like, at a given period of time following preparation of the formulation is significantly higher than the percent of active component present in another formulation at the same period of time following preparation of the formulation. In this case, the former formulation is said to possess increased stability relative to the latter formulation.

### B. Methods of Analysis

Crystallized samples of N-(4-chloro-3-methyl-5-isoxazolyl)-2-[2-methyl-4,5-(methylenedioxy)phenylacetyl]thiophene-3-sulfonamide, sodium salt, were analyzed by their XRPD, infrared absorption specta, Raman spectra, melting points, differential scanning calorimetry (DSC), thermogravimetry (TG), hot-stage microscopy and automated moisture sorption/desorption to determine their polymorphic forms (Forms A or B), hydrates and solvates (Form C).

1. XRPD

The XRPD analysis was carried out on a Shimadzu XRD-6000X-ray powder diffractometer using Cu Ka radiation. The instrument was equipped with a fine-focus X-ray tube. The tube power and amperage were set at 40 kV and 40 mA, respectively. The divergence and scattering slits were set at 1° and the receiving slit was set at 0.15 mm. Diffracted radiation was detected by a NaI scintillation detector. A theta-two theta continuous scan at 3°/min (0.4 sec/0.02° step) from 2.5° 2 theta to 40° 2 theta was used. A silicon standard was analyzed each day to check the instrument alignment. Each sample was prepared for analysis by placing it in a quartz sample holder. Three samples were analyzed with spinning (25 rpm) in order to reduce the effects of preferred orientation. The scan ran was adjusted to 0.5°/min to correct for the spin rate.

2. TG/IR

The TG/(R absorption were acquired on a TA Instruments TGA 2050 interfaced with a Nicolet model 560 Fourier transform IR spectrophotometer. This instrument was equipped with a globar source, a Ge/KBr beamsplitter, a deuterated triglycerine sulfate (DTGS) detector. The IR spectrophotometer was wavelength calibrated with polystyrene on the day of each use, while the TG was calibrated weekly, using nickel and alumel as standards. Approximately 5 mg of sample was weighed into a platinum pan and hewed from 20° C. to 150° C. at a rate of 20° C./min., with a helium purge. IR spectra were obtained in series with each spectrum representing 8 co-added scans at a resolution of 4 cm⁻¹. Volatiles were identified from a search of the HR Nicolet TGA vapor phase spectral library.

3. Raman Spectra

The Raman spectra were acquired on a Raman bench interfaced to a Nicolet Magna 860 FT-IR spectrophotometer. This instrument utilized an excitation wavelength of 1064 nm and approximately 0.5 W of Nd:YAG laser power. The spectra represent 32 or 64 co-added scans acquired at 4 cm⁻¹ resolution. The samples were prepared for analysis by placing the material in a glass tube and positioning this tube in the spectrophotometer. The spectrophotometer was calibrated (wavelength) with sulfur and cyclohexane at the time of use.

4. Differential Scanning Calorimetry (DSC)

The differential scanning calorimetry data was obtained on a TA Instruments Differential Scanning Calorimeter 2920. The calibration standard used was indium. Approximately 2 to 5 mg of a sample was placed into a DSC pan and the weight was accurately measured and recorded. The pan was hermetically sealed and a pinhole was used to allow for pressure release. The sample was heated under nitrogen at a rate of 10° C./min, up to a final temperature of 300° C. For studies of the glass transition temperature (T_{g}) of the amorphous material, the sample was heated under nitrogen at a rate of 10°/min, up to 125° C. The sample was held at this temperature for 15 minutes and then allowed to cool and equilibrate at 25° C. The sample was again heated at a rate of 10° C./min, up to 125° C., held at this temperature for 15 minutes and then coded and equilibrated at 25° C. for 15 minutes. The sample was then heated at 10° C./min, up to a final temperature of 200° C.

5. Thermogravimetric (TG) analysis

The thermogravimetric (TG) analysis of the samples was carried out on a TA Instruments Thermogravimetric Analyzer 2050 or 2950. The calibration standards used were nickel and Alumel™. Approximately 2 to 5 mg of a sample was placed in the pan, accurately weighed and inserted into the TG furnace. The sample was then heated in nitrogen at a rate of 10° C./min, up to a final temperature of 300° C.

6. Hot-Stage Microscopy

The hot-stage microscopy was carried out on a Kofler hot-stage mounted on a Leica Microscope. The temperature of the hot-stage was measured using a Testo 6000-903 thermocouple and a Testo 720 digital readout. Temperatures were calibrated using USP standards.

7. Moisture Sorption/Desorption

The moisture sorption/desorption data was collected on a VT SGA-100 moisture balance system. For sorption isotherms, a sorption range of 5 to 95% relative humidity (RH) and a desorption range of 95 to 5% RH in 10% RH increments were used for analysis. The sample was not dried prior to analysis. Equilibrium criteria used for analysis was less than 0.0100% weight change in 5 minutes with a maximum equilibration time of 3 hours if the weight criterion was not met. Data was not corrected for the initial moisture content of the samples.

8. Polymorph Screen

A polymorph screen was undertaken in an attempt to generate as many solid forms N-(4-chloro-3-methyl-5-isoxazolyl)-2-[2-methyl-4,5-(methylenedioxy)phenylacetyl] thiophene-3-sulfonamide, sodium salt, as possible. This technique involved the generation of solids under a variety of conditions and subsequent characterization by XRPD. Three distinct XRPD patterns representing three distinct forms, as well as an amorphous form, were found in the screen. The crystalline patterns are designated as Forms A, B and C. Form A was obtained from slow cooling of hot solutions, slurrying or from precipitation with an antisolvent. Form B was obtained from slow cooling of hot solutions and antisolvent crystallizations. Form C was obtained from antisolvent crystallizations from methyl t-butyl ether and appears to be the methyl t-butyl ether solvate of N-(4-chloro-3-methyl-5-isoxazolyl)-2-[2-methyl-4,5-(methylenedioxy)phenylacetyl]thiophene-3-sulfonamide, sodium salt. The amorphous material was produced from slow and fast evaporations of solutions.

9. Crystallization Procedures

Weighed samples of N-(4-chloro-3-methyl-5-isoxazolyl)-2-[2-methyl-4,5-(methylenedioxy)phenylacetyl]thiophene-3-sulfonamide, sodium salt (usually 30 mg) were treated with aliquots of a test solvent (reagent grade or HPLC grade) to provide 20 to 200 μL solutions. These solutions were sonicated and when all the solids dissolved (visual inspection), the solutions were filtered and left in an open vial under ambient conditions (fast evaporation) or were covered with aluminum foil containing pin holes (slow evaporation). Solids were removed by filtration, air-dried and analyzed by XRPD. Solid samples of this compound were also generated by rapidly cooling the above filtered, room temperature solutions to −78° C. (crash cool). Solids were removed by filtration, air-dried and analyzed by XRPD.

Weighed samples of N-(4-chloro-3-methyl-5-isoxazolyl)-2-[2-methyl-4,5-(methylenedioxy)phenylacetyl]thiophene-3-sulfonamide, sodium salt, were also treated with aliquots of a test solvent at elevated temperatures. These samples and solvents were heated on a hotplate held at either 45° C. or 80° C. and the resulting solution was rapidly filtered into a vial kept on the same hotplate. The heat source was turned off and the hotplate and vial were allowed to cool to ambient temperature (slow cool) and allowed to stand overnight. The presence or absence of undissolved solids was noted; if there were no solids present, or an amount of solid judged too small for XRPD analysis, the vial was placed in a refrigerator overnight. Again the presence or absence of undissolved solids was noted and if there were none, the vial was placed in a freezer overnight. Solids were removed by filtration, air-dried and analyzed by XRPD.

The solubilities of N-(4-chloro-3-methyl-5-isoxazolyl)-2-[2-methyl-4,5-(methylenedioxy)phenylacetyl]thiophene-3-sulfonamide, sodium salt, were estimated from experiments based on the total solvent used to give a solution. The actual solubilities may be greater than those calculated because of the use of too-large solvent aliquots or a slow rate of dissolution. If dissolution did not occur during the experiment the solubility is expressed as “less than”. If the solid dissolved before the whole aliquot of solvent was added the solubility is listed as “greater than”.

Antisolvent experiments were carried out by dissolving solid samples of N-(4-chloro-3-methyl-5-isoxazolyl)-2-[2-methyl-4,5-(methylenedioxy)phenylacetyl]thiophene-3-sulfonamide, sodium salt, in a test solvent and filtering the resulting solution into an antisolvent. If solids were formed it is termed a “crash crystallization”; and if solids formed after the solution was cooled or covered and left to stand, is termed a “precipitation”. If no solids immediately formed, the samples were left under ambient conditions until solids were seen. Any solids formed were removed by filtration, air-dried and analyzed by XRPD.

Slurry experiments were carried out by making saturated solutions of N-(4-chloro-3-methyl-5-isoxazolyl)-2-[2-methyl-4,5-(methylenedioxy)phenylacetyl]thiophene-3-sulfonamide, sodium salt, which contained excess solids. These slurries were agitated at ambient temperature for 3 days. The insoluble solids were removed by filtration, air-dried and analyzed by XRPD.

Vapor diffusion experiments were carried out by placing a saturated solution N-(4-chloro-3-methyl-5-isoxazolyl)-2-[2-methyl-4,5-(methylenedioxy)phenylacetyl]thiophene-3-sulfonamide, sodium salt, in a vial which was then placed in a larger vial containing an antisolvent. The larger vial was then sealed and kept at ambient temperature. Solids were removed by filtration, air-dried and analyzed by XRPD.

Liquid diffusion experiments were carried out by placing a saturated solution of N-(4-chloro-3-methyl-5-isoxazolyl)-2-[2-methyl-4,5-(methylenedioxy)phenylacetyl]thiophene-3-sulfonamide, sodium salt, in a vial and adding an immiscible antisolvent. The presence or absence of precipitated solids was noted. If solids formed, the solvents were decanted and the solids collected. If no solids formed, the vial was capped and left to stand at ambient temperature. Any solids formed were removed by filtration, air-dried and analyzed by XRPD.

A solid sample of N-(4-chloro-3-methyl-5-isoxazolyl)-2-[2-methyl-4,5-(methylenedioxy)phenylacetyl]thiophene-3-sulfonamide, sodium salt, was also generated by quickly cooling (−78° C.) a melt of this compound.

### C. Polymorphs A, B, C and an Amorphous Material

The solid forms obtained in the polymorph screen of N-(4-chloro-3-methyl-5-isoxazolyl)-2-[2-methyl-4,5-(methylenedioxy)phenylacetyl]thiophene-3-sulfonamide, sodium salt, are summarized in Tables 1 to 3. Three distinct XRPD patterns representing three distinct forms, designated as Forms A, B and C were found. Form A was obtained by slow cools, slurrying or antisolvent crystallizations. Form B was obtained from slow cooling of hot solutions and antisolvent crystallizations. Form C was obtained from antisolvent crystallizations from methyl t-butyl ether. The amorphous material was produced from slow and fast evaporations of solutions.

TABLE 1

| SOLVENT | METHOD^{a} | XRPD^{b} |
|---|---|---|
| Acetone | FE | LC |
| Acetone | SE | LC |
| Acetone | SC(45° C.) | IS |
| Acetonitrile | FE | amorphous, SS |
| Acetonitrile | SE | amorphous, SS |
| acetonitrile | FE(60° C.) | A |
| chloroform | slurry | A |
| dichloromethane | FE | NS |
| dichloromethane | SE | A |
| N,N-dimethyl- | FE | IS |
| formamide | | |
| N,N-dimethyl- | SE | IS |
| formamide | | |
| ethanol | FE | amorphous, SS |
| ethanol | SE | amorphous |
| ethanol | SC(60° C.) | A |
| ethyl acetate | SE | amorphous, SS |
| ethyl acetate | SE | IS |
| ethyl acetate | SC(60° C.) | A |
| hexanes | slurry | A |
| isopropanol | SE | amorphous |
| isopropanol | slurry | A |
| isopropyl acetate | slurry | A |
| methanol | FE | amorphous |
| methanol | FE | amorphous, SS |
| methanol | SC(60° C.) | amorphous + peak ~32 |
| | | °2theta |
| methyl t-butyl ether | slurry | A |
| methyl ethyl ketone | FE | LC, SS |
| methyl ethyl ketone | SE | IS |
| methyl ethyl ketone | FE(60° C.) | A |
| toluene | slurry | A |
| tetrahydrofuran | FE | IS |
| tetrahydrofuran | SE | A, SS |
| tetrahydrofuran | SC(60° C.) | amorphous |
| water | FE | amorphous |
| water^{c} | SE | amorphous |
| water | SC(60° C.) | amorphous |
| water | SC(40° C.) | amorphous |

^{a}FE = fast evaporation; SE = slow evaporation; SC = slow cool;
^{b}SS = small sample; IS = insufficient sample; NS = no solid; LC = low crystallinity;
^{c}The material was dissolved in water, cooled in a refrigerator and then warmed to room temperature.

Table 2 shows the results for the antisolvent recrystallizations.

TABLE 2

| SOLVENT | ANTISOLVENT | METHOD^{a} | XRPD^{b} |
|---|---|---|---|
| acetone | chloroform | PR | IS |
| acetone | dichloromethane | PR | B |
| acetone | isopropanol | PR | NS |
| acetone | methyl t-butyl ether | AC | B |
| acetone | toluene | AC | B |
| ethanol | chloroform | PR | NS |
| ethanol | dichloromethane | PR | NS |
| ethanol | hexanes | PR | IS |
| ethanol | isopropanol | PR | NS |
| ethanol | methyl t-butyl ether | PR | C |
| ethanol | methyl t-butyl ether | PR | B |
| ethanol | methyl t-butyl ether | PR | C(PO) + min B |
| ethanol | methyl t-butyl ether | PR | C(PO) |
| ethanol | methyl t-butyl ether | PR | B |
| ethanol | methyl t-butyl ether | PR | B |
| ethanol | methyl t-butyl ether | PR | B, SS |
| ethanol | methyl t-butyl ether | PR | C |
| ethanol | toluene | PR | NS |
| ethyl acetate | chloroform | PR | NS |
| ethyl acetate | dichloroform | PR | NS |
| ethyl acetate | hexanes | AC | B, SS |
| ethyl acetate | methyl t-butyl ether | PR | B, SS |
| ethyl acetate | toluene | PR | B(PO) |
| methanol | chloroform | PR | NS |
| methanol | dichloromethane | PR | NS |
| methanol | isopropanol | PR | NS |
| methanol | methyl t-butyl ether | AC | B |
| methanol | methyl t-butyl ether | AC | C(LC) |
| methanol | methyl t-butyl ether | AC | C + B |
| methanol | methyl t-butyl ether | AC | IS |
| methanol | methyl t-butyl ether | AC | IS |
| methanol | methyl t-butyl ether | AC | B, SS |
| methanol | methyl t-butyl ether | AC | B + min C |
| methanol | methyl t-butyl ether | AC | B, SS |
| methanol | toluene | PR | A |
| tetrahydrofuran | chloroform | AC | NS |
| tetrahydrofuran | dichloromethane | AC | NS |
| tetrahydrofuran | hexanes | AC | A |
| tetrahydrofuran | isopropanol | AC | NS |
| tetrahydrofuran | methyl t-butyl ether | AC | B |
| tetrahydrofuran | methyl t-butyl ether | AC | B |
| tetrahydrofuran | toluene | AC | IS |

^{a}PR = precipitation; AC = antisolvent crystallization;
^{b}PO = preferred orientation; IS = insufficient sample; SS = small sample; NS = no solid; LC = low crystallinity; A = polymorph A; B = polymorph B; C = polymorph C; Min = minor polymorph

Table 3 shows the results for the vapor diffusion experiments.

TABLE 3

| SOLVENT | ANTISOLVENT | HABIT^{a} |
|---|---|---|
| acetonitrile | isopropanol | NS |
| acetonitrile | dichloromethane | NS |
| ethanol | dichloromethane | NS |
| ethanol | isopropanol | NS |
| ethanol | hexanes | NS |
| ethyl acetate | chloroform | NS |
| ethyl acetate | isopropanol | rods |
| ethyl acetate | isopropanol | NS |
| ethyl acetate | isopropanol | rods |
| ethyl acetate | isopropyl acetate | NS |
| ethyl acetate | hexanes | needles |
| ethyl acetate | hexanes | needles |
| ethyl acetate | hexanes | unknown |
| methyl ethyl ketone | hexanes | unknown |
| methyl ethyl ketone | isopropanol | unknown |
| methyl ethyl ketone | isopropanol | unknown |

^{a}NS = no solid

a. Form A

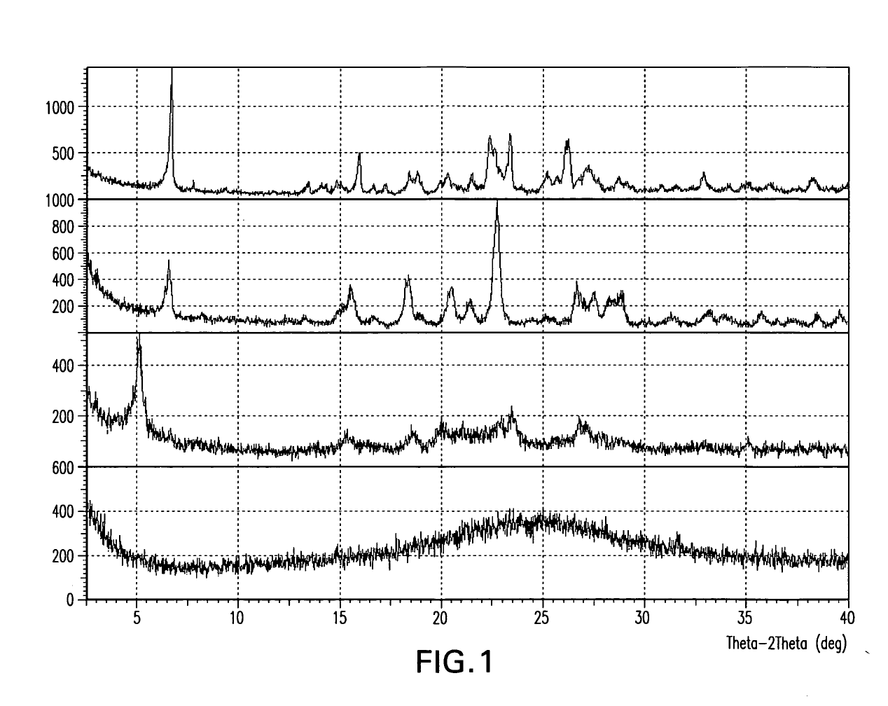

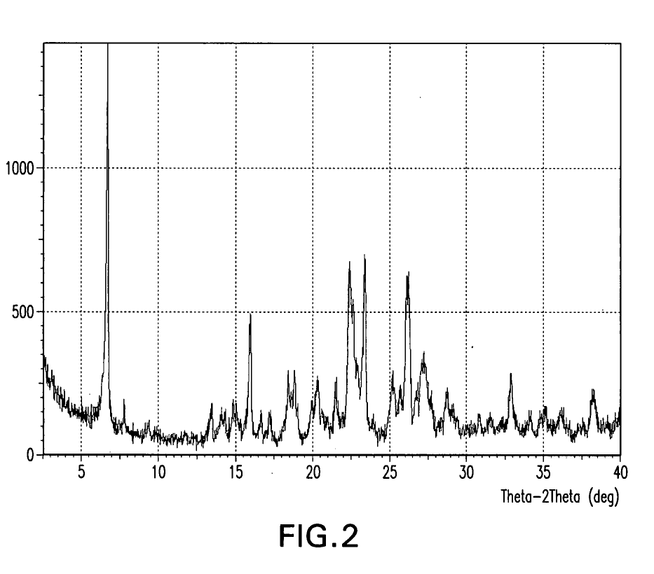

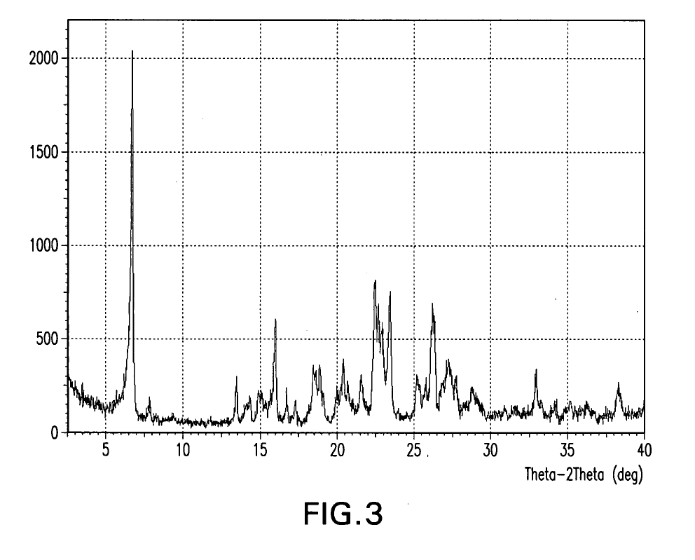

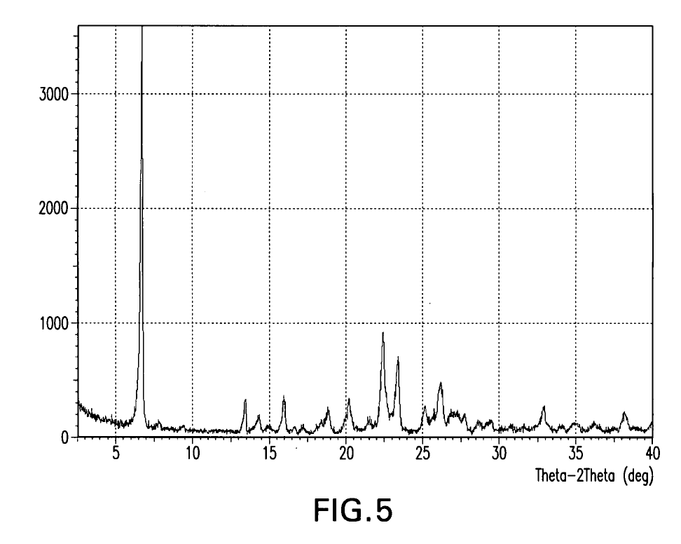

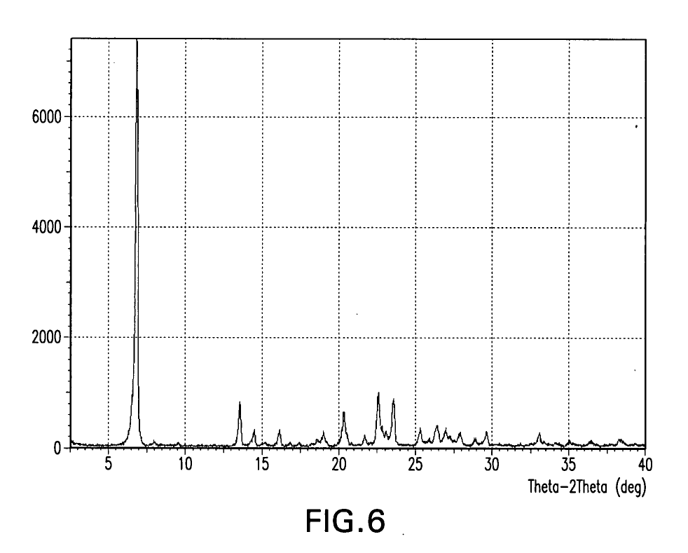

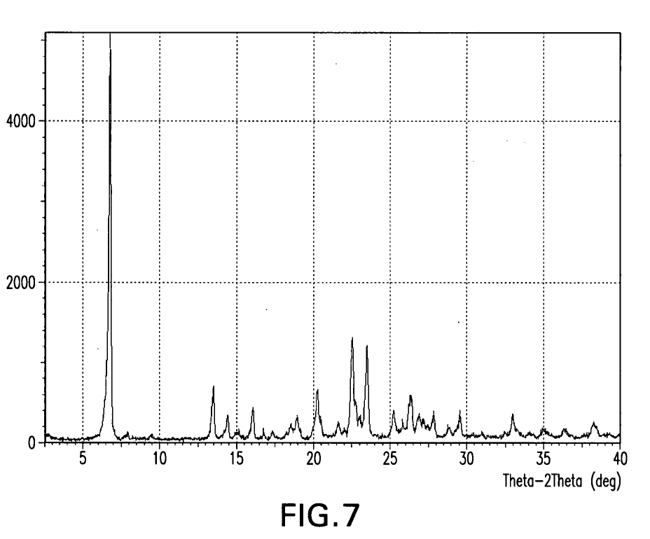

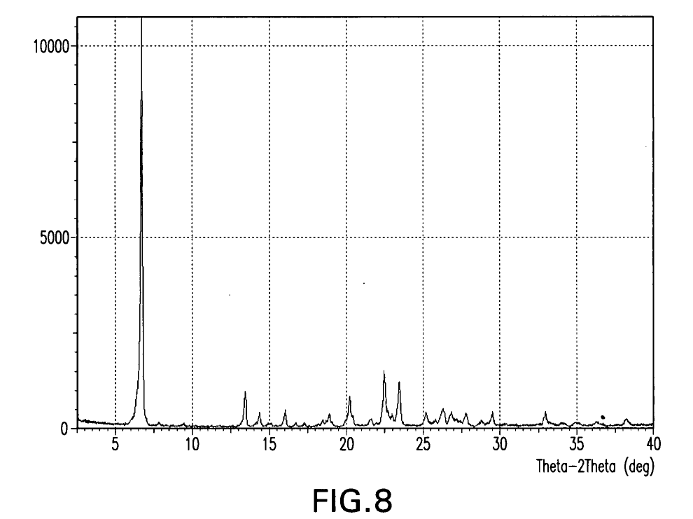

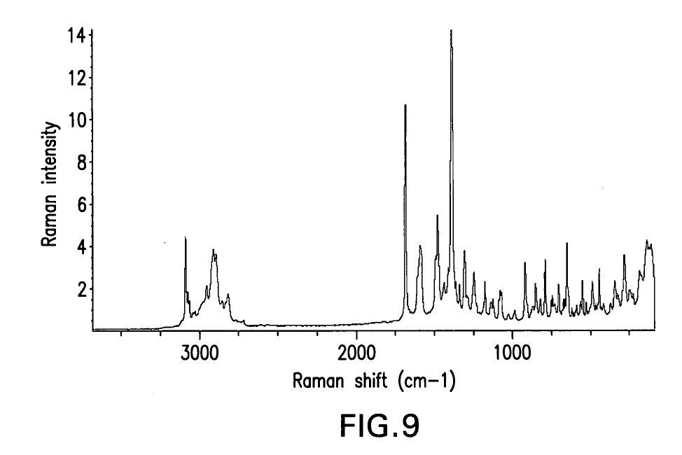

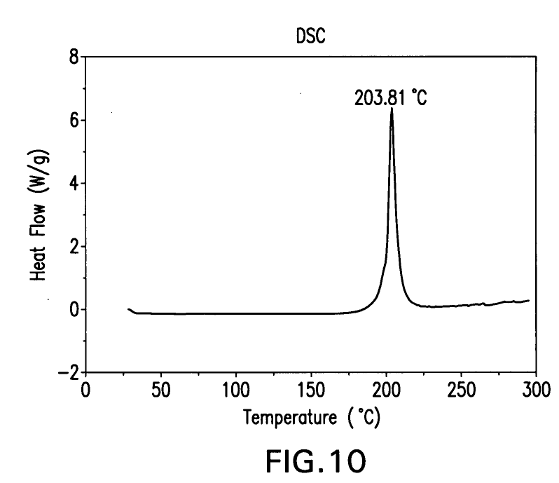

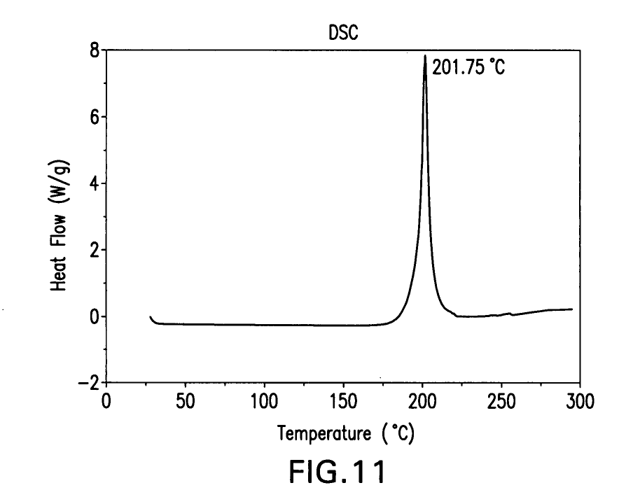

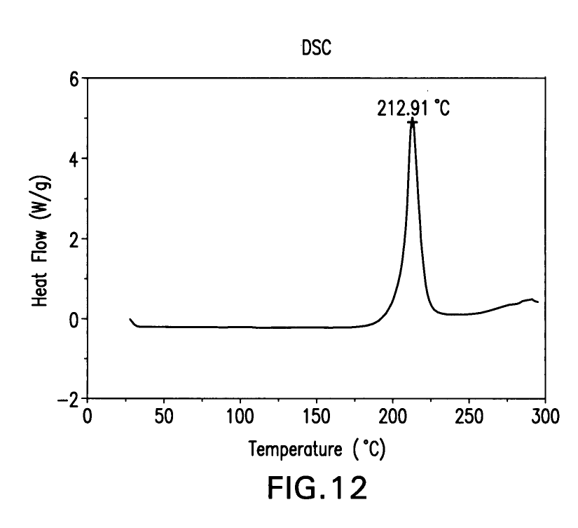

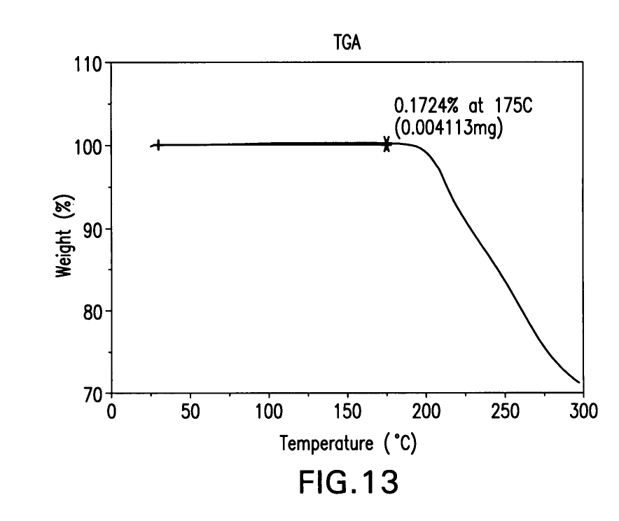

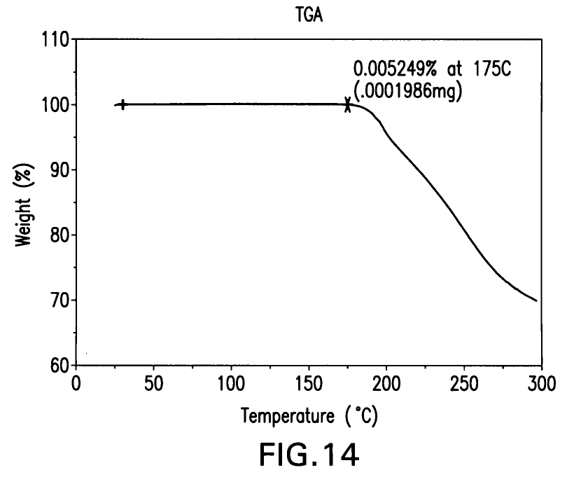

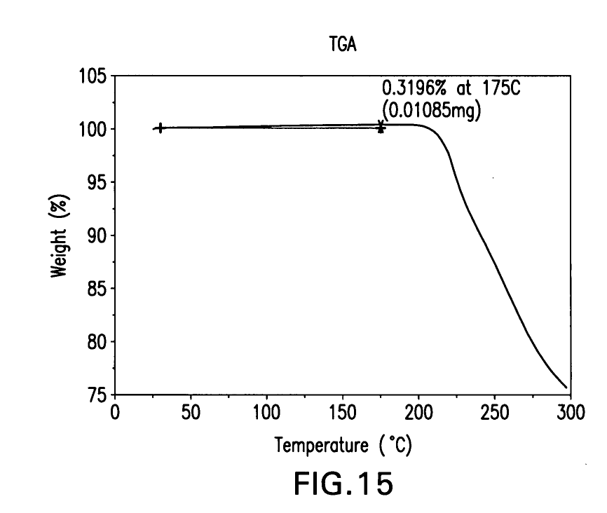

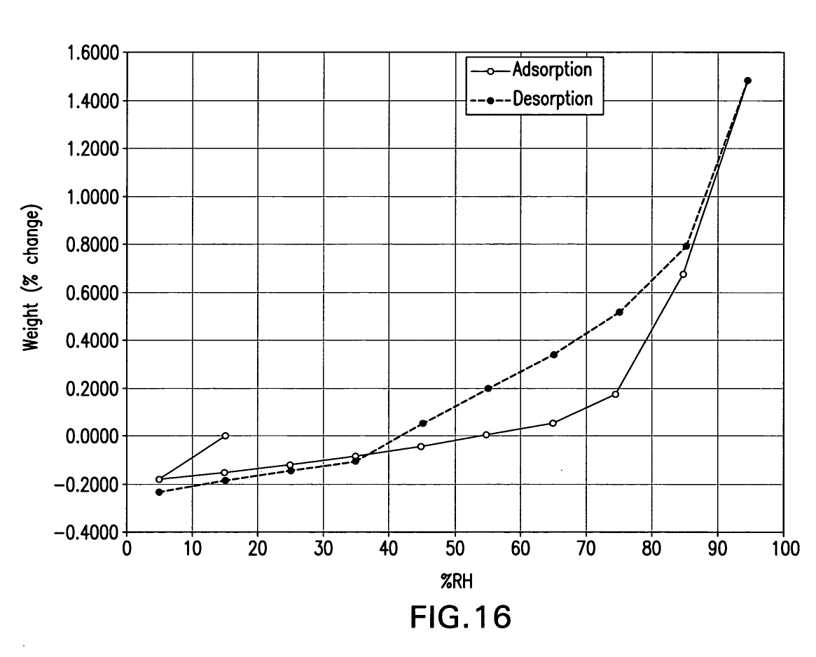

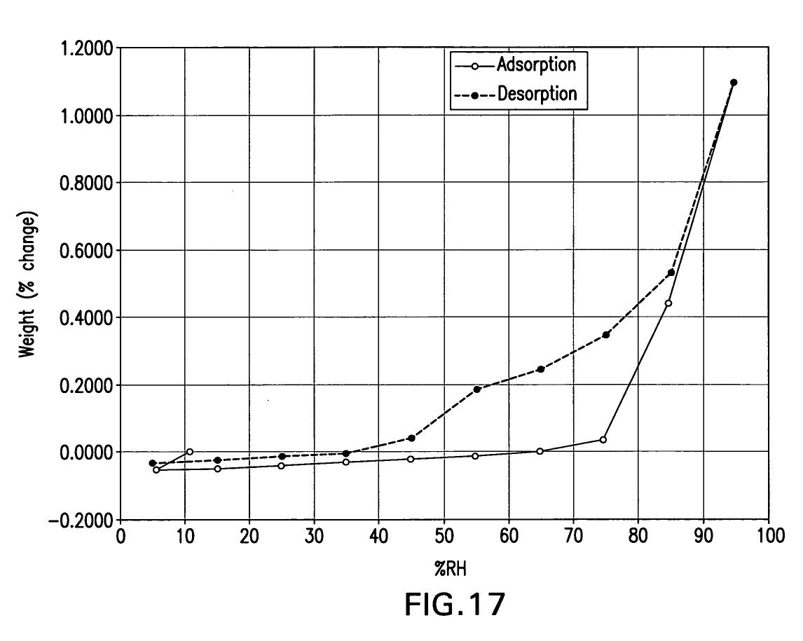

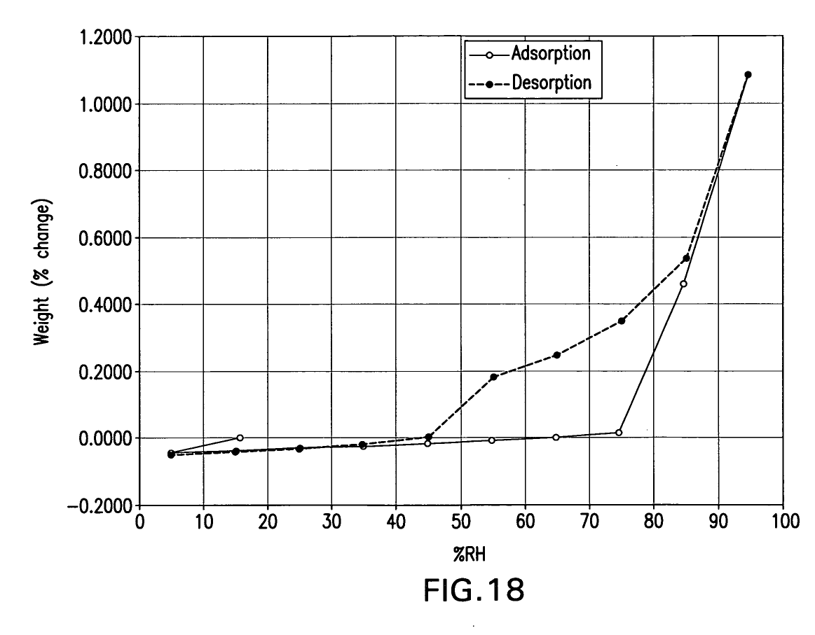

Form A of N-(4-chloro-3-methyl-5-isoxazolyl)-2-[2-methyl-4,5-(methylene-dioxy)phenylacetyl]thiophene-3-sulfonamide, sodium salt, was characterized using XRPD, DSC, TG, hotstage microscopy and moisture sorption/desorption and the data is shown in FIGS. 1 to 18, Table 4 (Hot stage studies), Table 5 (Moisture sorption/desorption data), Table 6 (XRPD peaks), Table 7 (peaks in Raman spectra) and Table 8 (peaks in IR spectra). Exothermic decomposition was seen around 200° C. and was confirmed by hotstage data. The TG curves show minimal weight change at 175° C. Moisture sorption/desorption data shows that samples of N-(4-chloro-3-methyl-5-isoxazolyl)-2-[2-methyl-4,5-(methylenedioxy)phenylacetyl]thiophene-3-sulfonamide, sodium salt, loses minimal weight at 5% RH, indicating that there are only a small amount of initial volatiles removed under low RH conditions. Samples gained less than 1.5% of their weight at 95% RH, which is less than the calculated weight gain (1.87%) for the formation of a hemihydrate. The majority of the weight is lost by 75% RH on the desorption curve, and the material returns to an unsolvated state upon equilibrium below 35% RH. The XRPD patterns of the samples after the experiment was completed indicate that the material was Form A. Form A material appears to be non-hygroscopic up to 75% RH, based on the moisture sorption/desorption data.

TABLE 4
Hot Stage Studies on TBC11251Na Lots

| Form | Lot No. | Observations (° C.)^{a} |
|---|---|---|
| A | Sample | 175 - start of decomp.; 199 - small amt. of opaque solids |
| | lot I | become biref.; 204 - solid decomposes, loses biref.; 243 - |
| | | turns brown |
| A | Sample | 193 - increase of biref.; 196-199 - starts to decompose, |
| | lot IV | turns brown; 206 - no biref. |
| A | Sample | 202-207 - decrease in biref.; 210-211 - decomposes, loses |
| | lot III | birefringence and turns brown |
| B | - | 188-191- increase of biref.; 194- some melt visible; |
| | | 200-203; decomposes, loses birefringence and turns |
| | | brown |

^{a}biref. = birefringent; decomp. = decomposition

TABLE 5
Summary of Moisture Sorption/Desorption Data for TBC11251Na Lots

| Lot No./Form^{a} | Moisture Balance Results |
|---|---|
| Sample lot I | 0.18% weight loss at 5% RH, |
| Form A | 1.48% total weight gain at 95% RH |
| Sample lot IV | 0.05% weight loss at 5% RH, |
| Form A | 1.10% total weight gain at 95% RH |
| Sample lot III | 0.04% weight loss at 5% RH, |
| Form A | 1.08% total weight gain at 95% RH |
| Form B | 0.13% weight loss at 5% RH, |
| | 0.98% total weight gain at 95% RH |
| Amorphous | 1.4% weight loss at 5% RH, |
| | 22.0.% total weight gain at 95% RH |

^{a}XRPD results on solid after moisture sorption experiment

TABLE 6
Peaks in the XRPD pattern of Forms A, B and C (degrees 2-theta)

| Form A | Form B | Form C |
|---|---|---|
| 6.72 | 6.6 | 5.14 |
| 7.8 | 8.2 | 13.9 |
| 9.38 | 12.28 | 15.36 |
| 13.46 | 13.22 | 18.52 |
| 14.32 | 15.12 | 20 |
| 14.86 | 15.52 | 20.76 |
| 15.96 | 16.58 | 22.8 |
| 16.66 | 18.38 | 23.48 |
| 17.2 | 18.94 | 24.1 |
| 18.42 | 20.52 | 24.4 |
| 18.82 | 21.5 | 26.78 |
| 19.94 | 22.72 | 27.06 |
| 20.32 | 24.44 | 32.98 |
| 21.56 | 25.14 | 35.08 |
| 22.38 | 26.66 | |
| 23.38 | 27.46 | |
| 25.2 | 28.26 | |
| 25.68 | 28.86 | |
| 26.22 | 29.88 | |
| 27.2 | 31.28 | |
| 28.78 | 33.16 | |
| 30.82 | 33.9 | |
| 31.48 | 35.76 | |
| 32.88 | 37.38 | |
| 34.18 | 38.42 | |
| 35.14 | 39.52 | |
| 36.18 | | |
| 38.28 | | |
| 39.9 | | |

TABLE 7
Peaks in the Raman Spectra of Forms A and B (cm⁻¹)

| Form A | Form B |
|---|---|
| 3105.9 | 3105.6 |
| 3090.3 | 3082 |
| 3082.1 | 3046.2 |
| 3046 | 2970 |
| 2970.3 | 2929.1 |
| 2927.6 | 2874.1 |
| 2909.9 | 2831 |
| 2871.5 | 2735.2 |
| 2833.2 | 1696.9 |
| 2733.6 | 1594.7 |
| 1697.4 | 1490.2 |
| 1602.1 | 1449.5 |
| 1503.5 | 1403.4 |
| 1489.8 | 1397.8 |
| 1449.7 | 1374.5 |
| 1423.3 | 1350.6 |
| 1402.2 | 1374.5 |
| 1375.3 | 1350.6 |
| 1350.8 | 1320.5 |
| 1318.2 | 1297.9 |
| 1257.2 | 1257.3 |
| 1188 | 1187.8 |
| 1149.3 | 1133.8 |
| 1137.1 | 1082.1 |
| 1090 | 998.3 |
| 1082.6 | 928.9 |
| 1036.2 | 866.1 |
| 997.8 | 835.4 |
| 930.3 | 802.9 |
| 882.1 | 763.4 |
| 864.4 | 755.5 |
| 835.5 | 744 |
| 803.5 | 714.8 |
| 763.8 | 686.4 |
| 755.8 | 662.2 |
| 743.2 | 632.5 |
| 716.6 | 600 |
| 685.7 | 576.6 |
| 674.3 | 563.4 |
| 661.5 | 540.7 |
| 632.3 | 499.6 |
| 619.8 | 455.6 |
| 602 | 427.7 |
| 576.1 | 356.6 |
| 562.7 | 292.4 |
| 540.3 | 260.6 |
| 499.8 | 152.8 |
| 478.1 | |
| 455 | |
| 428 | |
| 383.4 | |
| 354.5 | |
| 337.3 | |
| 292.1 | |
| 257.4 | |
| 237.2 | |
| 194.6 | |
| 146.8 | |
| 121 | |

TABLE 8
Peaks in the IR Spectra of Forms A and B (cm⁻¹)

| Form A | Form B |
|---|---|
| 3132.8 | 3135 |
| 3106.2 | 3105.4 |
| 3090.1 | 3081.5 |
| 3016.1 | 3047.2 |
| 2967.7 | 2970.2 |
| 2909.4 | 2947.5 |
| 2870.7 | 2915.5 |
| 2832 | 2885.1 |
| 1696.9 | 2827.5 |
| 1596.5 | 2777.4 |
| 1502.8 | 1696.7 |
| 1480.3 | 1594.9 |
| 1449.2 | 1505.2 |
| 1418.7 | 1482.9 |
| 1399.2 | 1450.4 |
| 1372.2 | 1404.6 |
| 1351.9 | 1397.1 |
| 1318.9 | 1371.5 |
| 1294.4 | 1351.1 |
| 1264.7 | 1321.1 |
| 1186.6 | 1297.4 |
| 1168.6 | 1260.5 |
| 1152.1 | 1186.9 |
| 1128.3 | 1151.8 |
| 1098.4 | 1125.7 |
| 1090 | 1100.3 |
| 1037.4 | 1089.8 |
| 996.2 | 1033.4 |
| 928.8 | 998 |
| 919.2 | 928 |
| 901.5 | 918.4 |
| 888.3 | 899.2 |
| 863.4 | 889.2 |
| 856.1 | 865.3 |
| 835.7 | 856.4 |
| 804.1 | 836.1 |
| 763.8 | 802.8 |
| 750.2 | 763.6 |
| 740 | 750.7 |
| 707 | 742 |
| 693.5 | 714.2 |
| 686.1 | 697.2 |
| | 686.4 |

Based on the characterization data, Form A appears to be an unsolvated, non-hygroscopic, crystalline material that decomposes above 200° C.

b. Form B

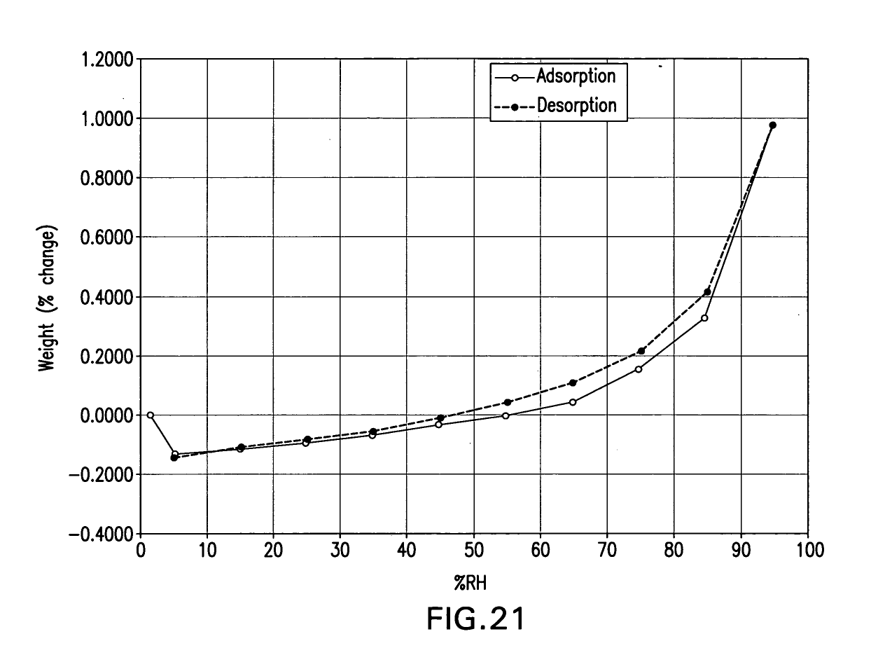

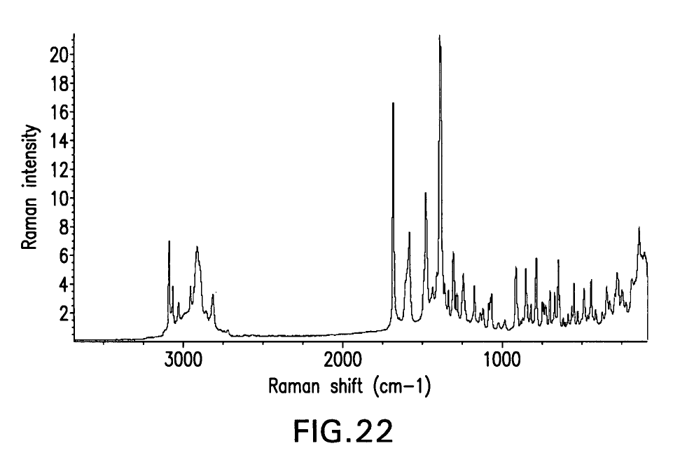

Form B of N-(4-chloro-3-methyl-5-isoxazolyl)-2-[2-methyl-4,5-(methylenedioxy)phenylacetyl]thiophene-3-sulfonamide, sodium salt, was usually obtained from antisolvent crystallizations and was characterized using XRPD, DSC, TG, hotstage microscopy and moisture sorption/desorption and the data is shown in FIGS. 1 and 19-22 and Table 4 (Hot stage studies), Table 5 (Moisture sorption/desorption data), Table 6 (XRPD peaks), Table 7 (peaks in Raman spectra) and Table 8 (peaks in IR spectra).

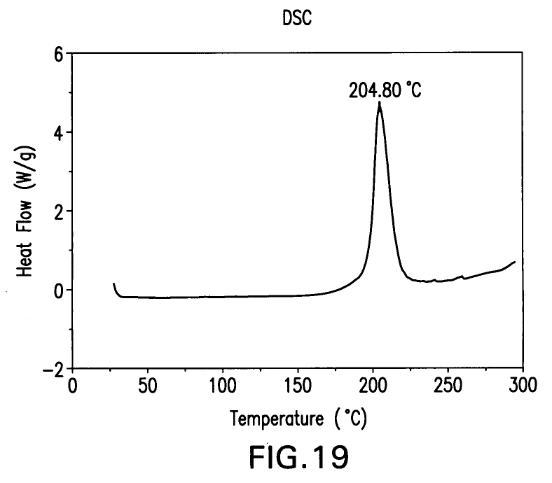

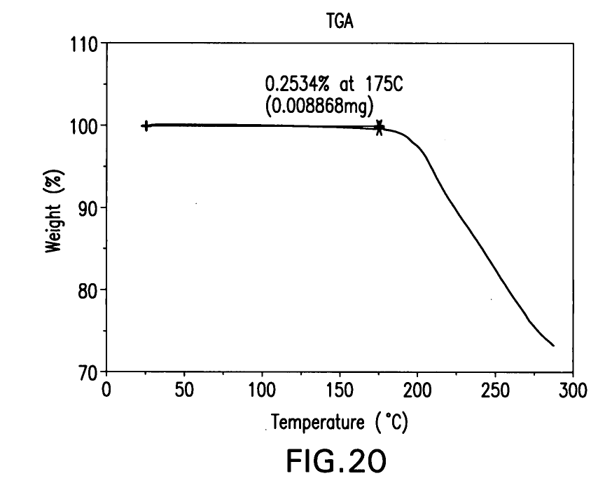

The thermal data for Form B is shown in FIGS. 19 and 20. The DSC exhibits a broad exotherm at 205° C. which is attributed to decomposition from hotstage data. The TG curve shows a minimal weight loss at 175° C. Form B loses and gains minimal weight during the moisture sorption/desorption experiment. The XRPD pattern collected on the sample after the experiment was completed indicates that the material was Form B. A sample of Form B was analyzed for it's sodium content (4.85%), which corresponds to the theoretical value (4.82%) for N-(4-chloro-3-methyl-5-isoxazolyl)-2-[2-methyl-4,5-(methylenedioxy)phenylacetyl]thiophene-3-sulfonamide, sodium salt, indicating that the salt was intact.

Based on the characterization data, Form B appears to be a nonsolvated crystalline material which decomposes around 203° C.

c. Form C

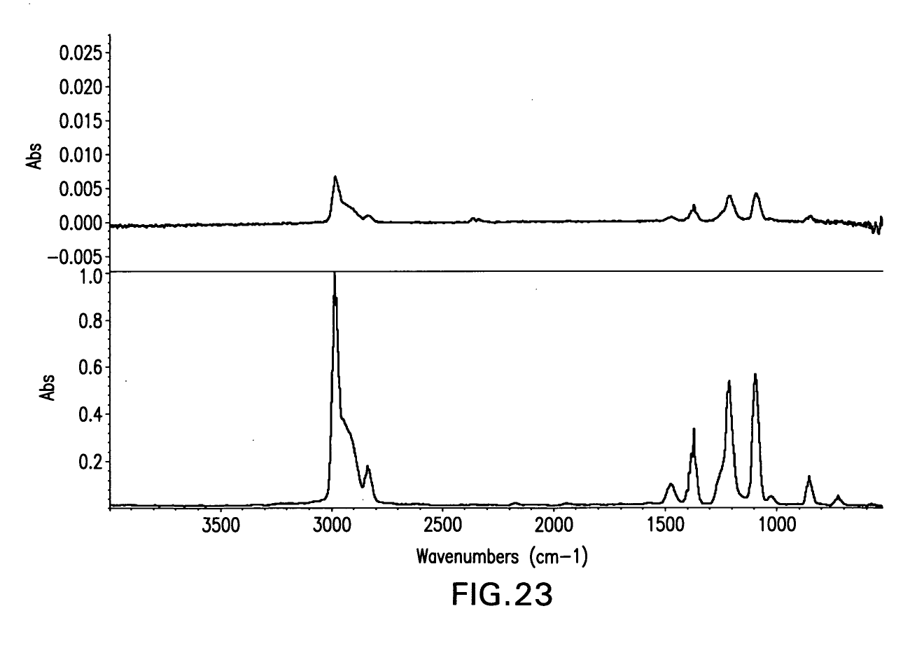

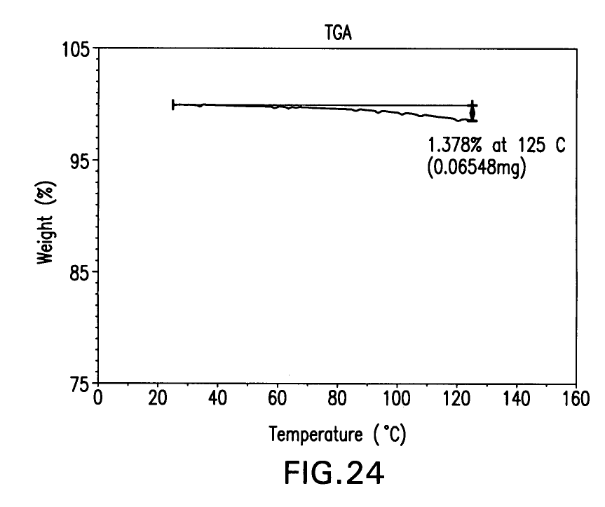

Form C of N-(4-chloro-3-methyl-5-isoxazolyl)-2-[2-methyl-4,5-(methylenedioxy)phenylacetyl]thiophene-3-sulfonamide, sodium salt, was obtained alone or in mixtures with Form B from antisolvent crystallizations using methanol or ethanol as a solvent and methyl-t-butyl ether as the antisolvent. Repeated crystallizations under the same conditions most often yielded Form B or mixtures of Forms B and C. Form C was characterized using XRPD, TG/IR and TG is shown in FIGS. 1 and 23-24 and Table 4.

TG data from a sample which contains Form C and a small amount of Form B shows a weight loss of 22.4% at 175° C., which is close to the calculated value for an octahydrate (calc. 23.2%) or a methyl-t-butyl etherate (21.7%, 3 molecules of solvent per 2 drug molecules) of N-(4-chloro-3-methyl-5-isoxazolyl)-2-[2-methyl-4,5-(methylenedioxy)-phenyl-acetyl]thiophene-3-sulfonamide, sodium salt. Elemental analysis of a similar sample gave a sodium content of 4.19%, which is slightly higher than that calculated for an octahydrate (3.7%) or the methyl-t-butyl etherate (3.8%) described above. A sample of Form C was analyzed using TG/IR and was found to contain methyl-t-butyl etherate, confirming that Form C is a methyl-t-butyl ether solvate of N-(4-chloro-3-methyl-5-isoxazolyl)-2-[2-methyl-4,5-(methylenedioxy)phenylacetyl]thiophene-3-sulfonamide, sodium salt. The material collected after the experiment was analyzed by XRPD and was found to remain in Form C.

### 2. Crystallization Studies

Crystallization studies and detailed processes for preparing polymorphs of N-(4-chloro-3-methyl-5-isoxazolyl)-2-[2-methyl-4,5-(methylenedioxy)phenylacetyl]thiophene-3-sulfonamide, sodium salt, in Forms A and B are described below. These studies demonstrate that these polymorphs can be selectively produced under appropriate conditions. Further, Form B and mixtures of Forms A and B, can be interconverted to Form A, suggesting that Form A is the more stable species.

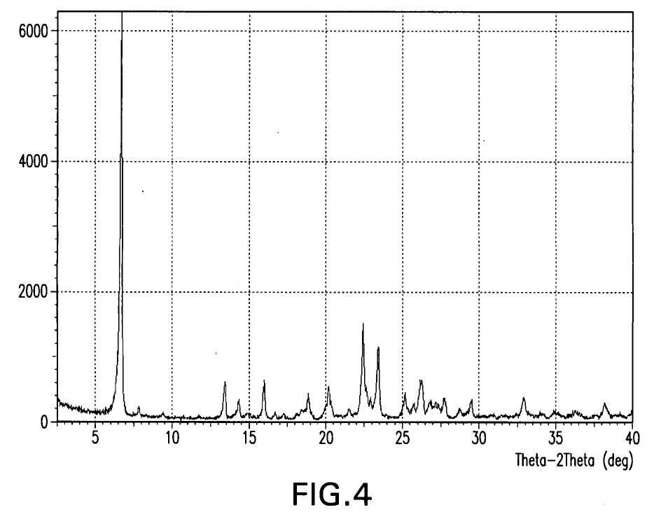

The XRPD patterns of the solid crystalline form of N-(4-chloro-3-methyl-5-isoxazolyl)-2-[2-methyl-4,5-(methylenedioxy)phenylacetyl]thiophene-3-sulfonamide, sodium salt, Forms A and B are shown in FIGS. 1 and 4, respectively. These XRPD patterns were used to identify the solid forms obtained from the crystallization and process studies described below.

### 3. Approximate Solubilities

Solubilities were estimated from experiments based on total solvent used to give a solution. The actual solubilities may be greater than those calculated because of the use of too-large solvent aliquots or a slow rate of dissolution. If dissolution did not occur during the experiment the solubility is expressed as “less than”. If the solid dissolved before the whole aliquot of solvent was added the solubility is listed as “greater than”.

The approximate solubilities of N-(4-chloro-3-methyl-5-isoxazolyl)-2-[2-methyl-4,5-(methylenedioxy)phenylacetyl]thiophene-3-sulfonamide, sodium salt, in Form A in various solvents at ambient temperature are summarized in Table 9. Form A was found to be most soluble in N,N-dimethylformamide (228 mg/mL), followed by methanol (160 mg/mL), acetone (96 mg/mL), tetrahydrofuran (86 mg/mL), ethanol (60 mg/mL), water (48 mg/mL) and methyl ethyl ketone (34 mg/mL). Form A of was poorly soluble in chloroform, dichloromethane and methyl t-butyl ether (<3 mg/mL).

TABLE 9

| SOLVENT^{a,b} | SOLUBILITY (mg/mL)^{c} |
|---|---|
| acetone | 96 |
| acetonitrile (ACN) | 25 |
| chloroform | <3 |
| dichloromethane (CH₂CI₂) | <3 |
| N,N-dimethylformamide (DMF) | >228 |
| ethanol (EtOH) | 60 |
| ethyl acetate (EtOAc) | 6 |
| hexanes | <8 |
| isopropanol (IPA) | <4 |
| methanol (MeOH) | 160 |
| methyl t-butyl ether (MTBE) | <3 |
| methyl ethyl ketone (MEK) | 34 |
| tetrahydrofuran (THF) | 86 |
| toluene | <7 |
| water | 48 |

^{a}The procedure used to determine the solubility of N-(4-chloro-3-methyl-5-isoxazolyl)-2-[2-methyl-4,5-(methylenedioxy)phenylacetyl]thiophene-3-sulfonamide, sodium salt, in various solvents was to add a test solvent in measured portions (usually 100 uL) to an accurately weighed sample with shaking, stirring or sonification at ambient temperature until a clear solution resulted.
^{b}Solvents are listed in alphabetical order.
^{c}Solubilities were calculated based on the total solvent used to give a solution. Actual solubilities may be greater due to the volume of the solvent portions utilized or to a slow rate of dissolution. Values are rounded to the nearest mg/mL.

4. Interconversion studies

The interconversion of Forms A and B were preformed using ethyl acetate and 95% isopropanol:water. Form A appears to be the more thermodynamically stable form in 95% isopropanol:water. The interconversions in ethyl acetate yielded a mixture of Forms A and B, which is probably due to the low solublility of the materials. These results are supported by the fact that Form B was formed by antisolvent crystallizations, which usually favors the formation of the less thermodynamically stable forms.

### D. Process for the Preparation of Polymorphs

Based on the interconversion studies in ethyl acetate, Form A appears to be the most stable form of N-(4-chloro-3-methyl-5-isoxazolyl)-2-[2-methyl-4,5-(methylenedioxy)phenylacetyl]thiophene-3-sulfonamide, sodium salt. Form A was obtained from slow cools, slurrying or antisolvent crystallizations. Form B was obtained from antisolvent crystallizations, which usually favor the formation of the less thermodynamically stable form. Form C was obtained from antisolvent crystallizations from methyl t-butyl ether and the amorphous material was obtained from slow and fast evaporations of solutions.

In certain embodiments, the process crystallization of sitaxsentan sodium provided herein produces a mixture of polymorphs A and B. In certain embodiments, the mixture contains polymorphs A and B in a ratio of about 60:40. In other embodiment the ratio of polymorph A to B is about or is greater than or is equal to about 65:35, 70:30, 75:25, 80:20, 85:15, 90:10, 92:8, 93:7, 94:6, 95:5, 98:2, 96:4, 97:3 or 99:1. In one embodiment, the process provided herein produces about 100% polymorph A. In one embodiment, the process provided herein produces about 100% polymorph B.

### E. Formulation and Administration of the Compositions

Formulations of the polymorphs are provided herein. The formulations are compositions designed for administration of the polymorphs provided herein. The compositions are suitable for oral and parental administrations. Such compositions include solutions, suspensions, tablets, dispersible tablets, pills, capsules, powders, sustained release formulations and any other suitable formation. In one embodiment, compositions will take the form of a pill or tablet. Methods for manufacture of tablets, capsules and other such formulations are known to those of skill in the art (see, e.g., Ansel, H. C. (1885) Forms, Introduction to Pharmaceutical Dosage Forms, 4th Edition, pp. 126-163).

In the formulations provided herein, effective concentrations of a polymorph or a mixture of polymorphs is(are) mixed with a suitable pharmaceutical carrier or vehicle. The concentrations of the polymorphs in the formulations are effective for delivery of an amount, upon administration, that ameliorates the symptoms of the endothelin-mediated disease. In certain embodiments, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound as a polymorph or a mixture of polymorphs, is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., U.S. Pat. No. 5,114,918 to Ishikawa et al.; EP A1 0 436 189 to BANYU PHARMACEUTICAL CO., LTD (Oct. 7, 1991); Borges, et al. (1989) Eur. J. Pharm. 165: 223-230; Filep et al. (1991) Biochem. Biophys. Res. Commun. 177: 171-176) and then extrapolated therefrom for dosages for humans.

The concentration of active compound polymorph or polymorph mixture in the drug composition will depend on absorption, inactivation and excretion rates of the active compound, the physicochemical characteristics of the compound, the dosage schedule, and amount administered as well as other factors known to those of skill in the art. For example, the amount that is delivered is sufficient to treat the symptoms of hypertension. The effective amounts for treating endothelin-mediated disorders are expected to be higher than the amount of the sulfonamide compound that would, be administered for treating bacterial infections.

In one embodiment, a therapeutically effective dosage should produce a serum concentration of active ingredient of from about 0.1 ng/ml to about 50-100 μg/ml. Pharmaceutical dosage unit forms are prepared to provide from about 20 mg to about 300 mg and from about 25 to about 200 mg, or from about 25 up to about 100 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, salts, esters, hydrates, solvates and prodrug forms. The derivative is selected to be a more stable form than the corresponding neutral compound.

Thus, effective concentrations or amounts of a polymorph or mixture of polymorphs provided herein or pharmaceutically acceptable derivatives thereof are mixed with a suitable pharmaceutical carrier or vehicle for systemic, topical or local administration to form pharmaceutical compositions.

The compositions are intended to be administered by an suitable route, which includes orally, parenterally, rectally and topically and locally depending upon the disorder being treated. For example, for treatment of ophthalmic disorders, such as glaucoma, formulation for intraocular also intravitreal injection is contemplated. In one embodiment, capsules and tablets are used for oral administration. Reconstitution of a lyophilized powder, prepared as described herein, may be used for parental administration. The compounds in liquid, semi-liquid or solid form and are formulated in a manner suitable for each route of administration. Modes of administration include parenteral and oral modes of administration.

Solutions or suspensions used parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO) using surfactants, such as tween, or dissolution in aqueous sodium bicarbonate. Derivatives of the compounds, such as prodrugs of the compounds may also be used in formulating effective pharmaceutical compositions.

Upon mixing or addition of the sodium salt of the sulfonamide compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds, particularly the pharmaceutically acceptable salts, such as the sodium salts, thereof. The pharmaceutically therapeutically active compounds and derivatives thereof are in certain embodiments formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa. 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen ethylene vinyl acetate, polyanhydrides, polyglycolic acid, polyorthoesters, polylactic acid and others. Methods for preparation of these formulations are known to those skilled in the art. In an embodiment, the contemplated compositions may contain 0.001%-100% active ingredient, in another embodiment 0.1-85%, in another embodiment 75-95%.

The compositions may be prepared with carriers that protect the compound against rapid elimination from the body, such as time release formulations or coatings.

The formulations may include other active compounds to obtain desired combinations of properties. The polymorphs may also be advantageously administered for therapeutic or prophylactic purposes together with another pharmacological agent known in general to be of value in treating one or more of the diseases or medical conditions referred to hereinabove, such as beta-adrenergic blocker (for example atenolol), a calcium channel blocker (for example nifedipine), an angiotensin converting enzyme (ACE) inhibitor (for example lisinopril), a diuretic (for example furosemide or hydrochlorothiazide), an endothelin converting enzyme (ECE) inhibitor (for example phosphoramidon), a neutral endopeptidase (NEP) inhibitor, an HMGCoA reductase inhibitor, a nitric oxide donor, an anti-oxidant, a vasodilator, a dopamine agonist, a neuroprotective agent, a steroid, a beta-agonist, an anti-coagulant, or a thrombolytic agent. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) 25-NF20 (2002). In general, lactose-free compositions contains active ingredients, a binder/filler, and a lubricant in pharmaceutically compatible and pharmaceutically acceptable amounts. Particular lactose-free dosage forms contain active ingredients, microcrystalline cellulose, pre-gelatinized starch, and magnesium stearate.

Further provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

1. Formulations for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art. Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the formulations are solid dosage forms, such as capsules or tablets. The tablets, pills, capsules, troches and the like can contain any of the following ingredients, or conjugates of a similar nature: a binder; a filler, a diluent; a disintegrating agent; a lubricant; a glidant; a sweetening agent; and a flavoring agent. Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions herein is typically present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions contain from about 0.5 to about 15 weight percent of disintegrant, or from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL®200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

If oral administration is desired, the polymorph or mixture of polymorphs could be provided in a composition that is formulated as enteric coating tablets, sugar-coated tablets, film-coated tablets or multiple compressed tablets. Enteric coating tablets protect the active ingredient from the acidic environment of the stomach. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges. The composition may also be formulated in combination with an antacid or other such ingredient.

When the dosage unit form is a capsule, it can contain, in addition to material of the above type, a liquid carrier such as a fatty oil. In a gelatin capsule, the solution or suspension containing sitaxsentan sodium, in for example propylene carbonate, vegetable oils or triglycerides, is encapsulated in the capsule. Such solutions, and the preparation and encapsulation thereof, are disclosed in U.S. Pat. Nos. 4,328,245; 4,409,239; and 4,410,545.

The active ingredient can also be mixed with other active materials which do not impair the desired action, or with materials that supplement the desired action, such as antacids, H2 blockers, and diuretics. Higher concentrations, up to about 98% by weight of the active ingredient may be included.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative.

An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia.

Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

The pharmaceutical compositions containing active ingredients in micellar form can be prepared as described in U.S. Pat. No. 6,350,458. Such pharmaceutical compositions are particularly effective in oral, nasal and buccal applications.

In certain embodiments, formulations include, but are not limited to, those containing a polymorph or mixture of polymorphs provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In certain embodiments, the polymorph or mixture of polymorphs is formulated as an oral tablet containing about 50 mg, about 75 mg, about 100 mg, about 150 mg, about 200 mg, about 250 mg, about 300 mg, about 350 mg of the active ingredient. The capsule can contain inactive ingredients, such as polyethylene glycol 400, polysorbate 20, povidone, and butylated hydroxyanisole. The capsule shell can contain gelatin, sorbitol special glycerin blend and titanium dioxide.

Exemplary Oral Tablet Formulations

In certain embodiments, the methods provided herein involve administration of oral tablets containing a polymorph or a mixture of polymorphs provided herein. In one embodiment, the oral tablet further contains a buffer. In one embodiment, the oral tablet further contains an antioxidant. In one embodiment, the oral tablet further contains a moisture barrier coating.

In some embodiments, the tablets contain excipients, including, but not limited to an antioxidant, such as sodium ascorbate, glycine, sodium metabisulfite, ascorbyl palmitate, disodium edetate (EDTA) or a combination thereof; a binding agent, such as hydroxypropyl methylcellulose; a diluent, such as lactose monohydrate, including lactose monohydrate fast flo (intragranular) and lactose monohydrate fast flo (extragranular) and microcrystalline cellulose and a buffer, such as phosphate buffer. The tablet can further contain one or more excipients selected from a lubricant, a disintegrant and a bulking agent.

In certain embodiments, the amount of sitaxsentan sodium in the oral tablet is from about 5% to about 40% of the total weight of the composition. In certain embodiments, the amount of sitaxsentan sodium is from about 7% to about 35%, 10% to about 30%, 12% to about 32%, 15% to about 30%, 17% to about 27%, 15% to about 25% of the total weight of the composition. In certain embodiments, the amount of sitaxsentan sodium is about 5%, 7%, 9%, 10%, 12%, 15%, 17%, 20%, 22%, 25%, 27%, 30%, 35% or 40% of the total weight of the composition. In certain embodiments, the amount of sitaxsentan sodium is about 20%.

In certain embodiments, the oral tablet contains about 10 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 280 mg, 300 mg or 350 mg of sitaxsentan sodium.

In certain embodiments, the tablets contain a combination of two antioxidants, such as ascorbyl palmitate and EDTA, disodium. In certain embodiments, the amount of ascorbyl palmitate in the formulation is in a range from about 0.05% to about 3% of the total weight of the tablet. In other embodiments, the amount of ascorbyl palmitate is in a range from about 0.07% to about 1.5%, 0.1% to about 1%, 0.15% to about 0.5% of the total weight of the tablet. In certain embodiments, the amount of ascorbyl palmitate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of ascorbyl palmitate in the formulation is about 0.2% of the total weight of the tablet.

In certain embodiments, the amount of ascorbyl palmitate in the oral tablet is from about 0.1 mg to about 5 mg, about 0.5 mg to about 4 mg, about 0.7 mg to about 3 mg or about 1 mg to about 2 mg. In certain embodiments, the amount of ascorbyl palmitate in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of ascorbyl palmitate in the formulation is about 1 mg.

In certain embodiments, the amount of EDTA, disodium in the formulation is in a range from about 0.05% to about 3% by weight of the total weight of the tablet. In other embodiments, the amount of EDTA, disodium is in a range from about 0.07% to about 1.5%, 0.1% to about 1%, 0.15% to about 0.5% of the total weight of the tablet. In certain embodiments, the amount of EDTA, disodium in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of EDTA, disodium in the formulation is about 0.2% of the total weight of the tablet.

In certain embodiments, the amount of EDTA, disodium in the oral tablet is from about 0.1 mg to about 5 mg, about 0.5 mg to about 4 mg, about 0.7 mg to about 3 mg or about 1 mg to about 2 mg. In certain embodiments, the amount of EDTA, disodium in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of EDTA, disodium in the oral tablet is about 1 mg.

In certain embodiments, the tablets contain a combination of diluents, such as microcrystalline cellulose (AVICEL PH 102), lactose monohydrate fast flo (intragranular) and lactose monohydrate fast flo (extragranular). In certain embodiments, the amount of lactose monohydrate fast flo (intragranular) in the oral tablet is from about 5% to about 30% of the total weight of the composition. In certain embodiments, the amount of lactose monohydrate fast flo (intragranular) is from about 7% to about 25%, from about 10% to about 20%, from about 13% to about 20% of the total weight of the tablet. In certain embodiments, the amount of lactose monohydrate fast flo (intragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (intragranular) is about 16.9% of the total weight of the tablet.

In certain embodiments, the amount of lactose monohydrate fast flo (intragranular) is from about 40 mg to about 100 mg, from about 45 mg to about 95 mg, from about 50 mg to about 90 mg. In certain embodiments, the amount of lactose monohydrate fast flo (intragranular) is about 40 mg, 45 mg, 50 mg, 55 mg, 60 mg, 65 mg, 70 mg, 75 mg, 80 mg, 81 mg, 82 mg, 83 mg, 83.5 mg, 84 mg, 84.1 mg, 84.2 mg, 84.3 mg, 84.4 mg, 84.5 mg, 84.6 mg, 84.7 mg, 85 mg, 85.5 mg, 90 mg, 90.5 mg or 100 mg. In certain embodiments, the amount of lactose monohydrate fast flo (intragranular) is about 84.3 mg.

In certain embodiments, the amount of lactose monohydrate fast flo (extragranular) is from about 7% to about 25%, from about 10% to about 20%, from about 13% to about 20% of the total weight of the tablet. In certain embodiments, the amount of lactose monohydrate fast flo (extragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (extragranular) is about 16.4% of the total weight of the tablet. In certain embodiments, the amount of lactose monohydrate fast flo (extragranular) in the oral tablet is from about 40 mg to about 100 mg, from about 45 mg to about 95 mg, from about 50 mg to about 90 mg. In certain embodiments, the amount of lactose monohydrate fast flo (extragranular) is about 40 mg, 45 mg, 50 mg, 55 mg, 60 mg, 65 mg, 70 mg, 75 mg, 80 mg, 81 mg, 81.3 mg, 81.5 mg, 81.8 mg, 82 mg, 82.3 mg, 82.5 mg, 82.7 mg, 83 mg, 83.5 mg, 84 mg, 85 mg, 85.5 mg, 90 mg, 90.5 mg or 100 mg. In certain embodiments, the amount of lactose monohydrate fast flo (intragranular) is about 82 mg.

In certain embodiments, the amount of microcrystalline cellulose (Avicel PH 102) in the oral tablet is from about 10% to about 50% of the total weight of the composition. In certain embodiments, the amount of microcrystalline cellulose (Avicel PH 102) is from about 15% to about 45%, from about 20% to about 43%, from about 25% to about 40% of the total weight of the tablet. In certain embodiments, the amount of microcrystalline cellulose (Avicel PH 102) is about 15%, 17%, 20%, 23%, 25%, 27%, 30%, 32%, 34%, 35%, 37%, 40%, 42%, 45% or 50% of the total weight of the tablet. In certain embodiments, the amount of microcrystalline cellulose (Avicel PH 102) is about 35% of the total weight of the tablet.

In certain embodiments, the amount of microcrystalline cellulose (Avicel PH 102) in the oral tablet is from about 130 mg to about 300 mg. In certain embodiments, the amount of microcrystalline cellulose (Avicel PH 102) is from about 140 mg to about 275 mg or about 150 mg to about 250 mg. In certain embodiments, the amount of microcrystalline cellulose (Avicel PH 102) is about 150 mg, 160 mg, 165 mg, 170 mg, 175 mg, 180 mg, 185 mg, 190 mg or 200 mg. In certain embodiments, the amount of microcrystalline cellulose (Avicel PH 102) in the oral tablet is about 175 mg.

In certain embodiments, the binding agent is hydroxypropyl methylcellulose (E-5P). In certain embodiments, the amount of hydroxypropyl methylcellulose (E-5P) in the tablet is from about 0.5% to about 20% of the total weight of the composition. In certain embodiments, the amount of hydroxypropyl methylcellulose (E-5P) is from about 1% to about 15%, from about 2% to about 10%, from about 3% to about 8% of the total weight of the tablet. In certain embodiments, the amount of hydroxypropyl methylcellulose (E-5P) is about 1%, 2%, 3%, 4%, 5%, 6%, 7%, 8%, 9% or 10% of the total weight of the tablet. In certain embodiments, the amount of hydroxypropyl methylcellulose (E-5P) is about 5% of the total weight of the tablet.

In certain embodiments, the amount of hydroxypropyl methylcellulose (E-5P) in the tablet is from about 5 mg to about 50 mg, about 10 mg to about 40 mg or about 15 mg to about 30 mg. In certain embodiments, the amount of hydroxypropyl methylcellulose (E-5P) in the tablet is about 10 mg, 15 mg, 20 mg, 22 mg, 25 mg, 27 mg, 30 mg, 35 mg or about 40 mg. In certain embodiments, the amount of hydroxypropyl methylcellulose (E-5P) in the tablet is about 25 mg.

The formulations of sitaxsentan sodium provided herein are stable at neutral pH. In certain embodiments, buffer agent mixture, such as sodium phosphate monobasic monohydrate and sodium phosphate dibasic anhydrous is used to improve drug stability in the tablets. In certain embodiments, the amount of sodium phosphate, monobasic monohydrate ranges from about 0.05% to about 3% by weight of the total weight of the tablet. In other embodiments, the amount of sodium phosphate, monobasic monohydrate is in a range from about 0.07% to about 1.5%, 0.1% to about 1%, 0.15% to about 0.5% of the total weight of the tablet. In certain embodiments, the amount of sodium phosphate, monobasic monohydrate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1. %. In certain embodiments, the amount of sodium phosphate, monobasic monohydrate in the formulation is about 0.1% of the total weight of the tablet.

In certain embodiments, the amount of sodium phosphate, monobasic monohydrate in the oral tablet is from about 0.1 mg to about 3 mg, about 0.2 mg to about 2.5 mg, about 0.5 mg to about 2 mg or about 0.6 mg to about 1 mg. In certain embodiments, the amount of sodium phosphate, monobasic monohydrate in the oral tablet is about 0.1 mg, 0.2 mg, 0.3 mg, 0.4 mg, 0.5 mg, 0.6 mg, 0.7 mg, 0.8 mg, 0.9 mg or about 1 mg. In certain embodiments, the amount of sodium phosphate, monobasic monohydrate in the oral tablet is about 0.6 mg.

In certain embodiments, the amount of sodium phosphate, dibasic anhydrous ranges from about 0.05% to about 3% by weight of the total weight of the tablet. In other embodiments, the amount of sodium phosphate dibasic is in a range from about 0.07% to about 1.5%, 0.1% to about 1%, 0.15% to about 0.5% of the total weight of the tablet. In certain embodiments, the amount of sodium phosphate dibasic in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1. %. In certain embodiments, the amount of sodium phosphate dibasic in the formulation is about 0.2% of the total weight of the tablet.

In certain embodiments, the amount of sodium phosphate, dibasic anhydrous in the oral tablet is from about 0.1 mg to about 3.5 mg, about 0.5 mg to about 2.5 mg, or about 0.7 mg to about 2 mg. In certain embodiments, the amount of sodium phosphate, dibasic anhydrous in the oral tablet is about 0.1 mg, 0.3 mg, 0.5 mg, 0.7 mg, 0.9 mg, 1 mg, 1.1 mg, 1.3 mg, 1.5 mg, 1.7 mg or 2 mg. In certain embodiments, the amount of sodium phosphate, dibasic anhydrous in the oral tablet is about 1.1 mg.

In certain embodiments, the tablet contains disintegrants, such as Sodium Starch Glycoloate (intragranular) and Sodium Starch Glycoloate (extragranular). In certain embodiments, the amount of Sodium Starch Glycoloate (intragranular) in the tablet is from about 0.1% to about 10% of the total weight of the composition. In certain embodiments, the amount of Sodium Starch Glycoloate (intragranular) is from about 0.5% to about 8%, from about 1% to about 5%, from about 2% to about 4% of the total weight of the tablet. In certain embodiments, the amount of Sodium Starch Glycoloate (intragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of Sodium Starch Glycoloate (intragranular) is about 2.5% of the total weight of the tablet. In certain embodiments, the amount of Sodium Starch Glycoloate (intragranular) is from about 30 mg to about 5 mg, from about 20 mg to about 10 mg, from about 15 to about 10 mg. In certain embodiments, the amount of Sodium Starch Glycoloate (intragranular) is about 5 mg, 7 mg, 10 mg, 11 mg, 11.5 mg, 12 mg, 12.5 mg, 13 mg, 15 mg or 20 mg. In certain embodiments, the amount of Sodium Starch Glycoloate (intragranular) is about 12.5 mg.

In certain embodiments, the amount of Sodium Starch Glycoloate (extragranular) in the tablet is from about 0.1% to about 10% of the total weight of the composition. In certain embodiments, the amount of Sodium Starch Glycoloate (extragranular) is from about 0.5% to about 8%, from about 1% to about 5%, from about 2% to about 4% of the total weight of the tablet. In certain embodiments, the amount of Sodium Starch Glycoloate (extragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of Sodium Starch Glycoloate (extragranular) is about 2.5% of the total weight of the tablet. In certain embodiments, the amount of Sodium Starch Glycoloate (extragranular) is from about 30 mg to about 5 mg, from about 20 mg to about 10 mg, from about 15 to about 10 mg. In certain embodiments, the amount of Sodium Starch Glycoloate (extragranular) is about 5 mg, 7 mg, 10 mg, 11 mg, 11.5 mg, 12 mg, 12.5 mg, 13 mg, 15 mg or 20 mg. In certain embodiments, the amount of Sodium Starch Glycoloate (extragranular) is about 12.5 mg.

In certain embodiments, the tablet contains a lubricant, such as magnesium stearate. In certain embodiments, the amount of magnesium stearate in the tablet is from about 0.1% to about 8% of the total weight of the composition. In certain embodiments, the amount of magnesium stearate is from about 0.5% to about 6%, from about 0.7% to about 5%, from about 1% to about 4% of the total weight of the tablet. In certain embodiments, the amount of magnesium stearate is about 0.5%, 0.7%, 1%, 1.2%, 1.5%, 1.7%, 2%, 2.5% or 3% of the total weight of the tablet. In certain embodiments, the amount of magnesium stearate is about 2.5% of the total weight of the tablet. In certain embodiments, the amount of magnesium stearate in the tablet is from about 15 mg to about 1 mg. In certain embodiments, the amount of magnesium stearate is from about 10 mg to about 3 mg or from about 7 mg to about 5 mg. In certain embodiments, the amount of magnesium stearate is about 3 mg, 4 mg, 4.5 mg, 5 mg, 6 mg, 7 mg, 8 mg, 9 mg or 10 mg. In certain embodiments, the amount of magnesium stearate is about 5 mg.

The tablet formulations provided herein contain, in one embodiment, a moisture barrier coating. Suitable coating materials are known in the art and include, but are not limited to coating agents either of cellulose origin such as cellulose phthalate (Sepifilm, Pharmacoat), or of polyvinyl origin of Sepifilm ECL type, or of saccharose origin such as the sugar for sugar-coating of Sepisperse DR, AS, AP OR K (coloured) type, such as Sepisperse Dry 3202 Yellow, Blue Opadry, Eudragit EPO and Opadry AMB. The coating serves as a moisture barrier to hinder oxidation of sitaxsentan sodium. In certain embodiments, the coating materials are Sepifilm LP014/Sepisperse Dry 3202 Yellow (Sepifilm/Sepisperse) (3/2 wt/wt) at from about 1 to about 7% or about 4% tablet weight gain. In certain embodiments, the coating material is Sepifilm LP014/Sepisperse Dry 3202 Yellow (Sepifilm/Sepisperse). In certain embodiments, the Sepifilm/Sepisperse ratio is 1:2, 1:1 or 3:2 wt/wt. In certain embodiments, the Sepifilm/Sepisperse coating is at about 1%, 2%, 3%, 4%, 5%, 6% or 7% tablet weight gain. In certain embodiments, the Sepifilm/Sepisperse coating is at about 1.6% tablet weight gain. In certain embodiments, the Sepisperse Dry 3202 (yellow) is at about 0.5%, 0.8%, 1%, 1.3%, 1.6%, 2%, 2.4%, 2.5%, 3% or 4% tablet weight gain. In certain embodiments, the Sepisperse Dry 3202 (yellow) is at about 2.4% tablet weight gain. In certain embodiments, the Sepisperse Dry 3202 (yellow) is at about 1 mg, 3 mg, 5 mg, 6 mg, 7 mg, 8 mg, 9 mg, 10 mg, 13 mg 15 mg or 20 mg per tablet. In certain embodiments, the Sepisperse Dry 3202 (yellow) is at about 8 mg per tablet. In certain embodiments, the Sepifilm LP 014 is at about 0.5%, 1%, 1.5%, 2%, 2.2%, 2.4%, 2.6%, 3%, 3.5% or 4% tablet weight gain. In certain embodiments, the Sepifilm LP 014 is at about 2.4% tablet weight gain. In certain embodiments, the Sepifilm LP 014 is at about 5 mg, 7 mg, 9 mg, 10 mg, 11 mg, 12 mg, 13 mg, 15 mg, 17 mg or 20 mg per tablet. In certain embodiments, the Sepifilm LP 014 coating is at about 12 mg per tablet.

In certain embodiments, the tablet contains sitaxsentan sodium, microcrystalline cellulose, lactose monohydrate fast flo (intragranular), lactose monohydrate fast flo (extragranular), hydroxypropyl methylcellulose E-5P, ascorbyl palmitate, disodium EDTA, sodium phosphate monobasic, monohydrate, sodium phosphate dibasic, anhydrous, Sodium Starch Glycoloate (intragranular), Sodium Starch Glycoloate (extragranular), magnesium stearate and a coating of Sepifilm LP014/Sepisperse Dry 3202 Yellow.

In certain embodiments, the tablet contains about 20% sitaxsentan sodium, about 35% microcrystalline cellulose, about 16.9% lactose monohydrate fast flo (intragranular), about 16.4% lactose monohydrate fast flo (extragranular), about 5.0% hydroxypropyl methylcellulose E-5P, about 0.2% ascorbyl palmitate, about 0.2% disodium (EDTA), about 0.1% sodium phosphate monobasic, monohydrate, about 0.2% sodium phosphate dibasic, anhydrous, about 2.5% Sodium Starch Glycoloate (extragranular), about 2.5% Sodium Starch Glycoloate (intragranular) and about 1% magnesium stearate. The tablet further contains a coating of Sepifilm LP014 at about 2.4% weight gain and Sepisperse Dry 3202 Yellow at about 1.6% weight gain.

In certain embodiments, the oral tablet provided herein is a 500 mg tablet that contains about 100 mg sitaxsentan sodium, about 1.0 mg ascorbyl palmitate, about 1.0 mg disodium edetate (EDTA), about 25 mg hydroxypropyl methylcellulose E-5P, about 84.3 lactose monohydrate fast flo (intragranular), about 82 mg lactose monohydrate fast flo (extragranular), about 175 mg microcrystalline cellulose, about 0.6 mg sodium phosphate monobasic, monohydrate, about 1.1 mg sodium phosphate dibasic, anhydrous, about 12.5 mg Sodium Starch Glycoloate (extragranular), about 12.5 mg Sodium Starch Glycoloate (intragranular), about 5 mg magnesium stearate, non-bovine and about 192.5 mg purified water. The tablet further contains a coating of Sepifilm LP014 at about 12 mg and Sepisperse Dry 3202 Yellow at about 8 mg.

b. Sustained Release Dosage Form

Polymorphs provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the polymorph or mixture of polymorphs may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

c. Parenteral Administration

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of sitaxsentan sodium is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active ingredient is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, or more than 1% w/w of sitaxsentan to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The polymorphs or mixture of polymorphs may be suspended in micronized or other suitable form or may be derivatized to produce a more soluble active product or to produce a prodrug. The form of the resulting mixture depends upon a number of factors, including the intended mode of administration and the solubility of sitaxsentan sodium in the selected carrier or vehicle. The effective concentration is sufficient for ameliorating the symptoms of the condition and may be empirically determined.

d. Lyophilized Powders

Also provided herein are lyophilized powders, which can be reconstituted for administration as solutions, emulsions and other mixtures. They may also be reconstituted and formulated as solids or gels.

The sterile, lyophilized powder is prepared by dissolving the active ingredient, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-350 mg, or 100-300 mg) or multiple dosages of sitaxsentan sodium. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg, or about 9-30 mg of lyophilized powder, is added per mL of sterile water or other suitable carrier. The precise amount depends upon the selected conjugate. Such amount can be empirically determined.

Exemplary Lyophilized Formulations

In certain embodiments, provided herein are stable lyophilized powders of sitaxsentan sodium. The lyophilized powder contains an antioxidant, a buffer and a bulking agent. In the lyophilized powders provided herein, the amount of sitaxsentan sodium present is in a range from about 25% to about 60% by total weight of the lyophilized powder. In certain embodiments, the amount of sitaxsentan sodium is from about 30% to about 50% or about 35% to about 45% by total weight of the lyophilized powder. In certain embodiments, the amount of sitaxsentan sodium is about 30%, 33%, 35%, 37%, 40%, 41%, 43%, 45%, 47%, 50%, 53%, 55% or 60% by total weight of the lyophilized powder. In one embodiment, the amount of sitaxsentan sodium in the lyophilized powder is about 41% by total weight of the lyophilized powder.

In certain embodiments, the lyophilized powder contains an antioxidant, such as sodium sulfite, sodium bisulfite, sodium metasulfite, monothioglycerol, ascorbic acid or a combination thereof. In one embodiment, the antioxidant is monothioglycerol. In one embodiment, the antioxidant is a combination of ascorbic acid, sodium sulfite and sodium bisulfite. In certain embodiments, the lyophilized formulations provided herein have improved stability upon reconstitution as compared to the known lyophilized formulations of sitaxsentan sodium (see WO 98/49162).

In certain embodiments, the antioxidant is monothioglycerol. In certain embodiments, the monothioglycerol is present in an amount ranging from about 10% to about 30% by total weight of the lyophilized powder. In certain embodiments, the monothioglycerol is present in an amount ranging from about 12% to about 25% or about 15% to about 20% by total weight of the lyophilized powder. In certain embodiments, the amount of monothioglycerol in the lyophilized powder is about 10%, 12%, 14%, 15%, 15.5%, 16%, 16.2%, 16.4%, 16.8%, 17%, 17.5%, 19%, 22%, 25% or 30% by total weight of the lyophilized powder. In certain embodiments, the amount of monothioglycerol is about 16.4% by total weight of the lyophilized powder.

In certain embodiments, the sodium sulfite is present in an amount from about 1% to about 6% by total weight of the lyophilized powder. In other embodiments, the sodium sulfite is present in an amount from about 1.5% to about 5% or about 2% to about 4%. In certain embodiments, the amount of sodium sulfite is about 1%, 1.5%, 2%, 2.5%, 3%, 3.3%, 3.5%, 3.8%, 4%, 4.5% or 5% by total weight of the lyophilized powder. In one embodiment, the amount of sodium sulfite is about 3.3% by total weight of the lyophilized powder.

In certain embodiments, the ascorbic acid is present in an amount from about 1% to about 6% by total weight of the lyophilized powder. In other embodiments, the ascorbic acid is present in an amount from about 1.5% to about 5% or about 2% to about 4%. In certain embodiments, the amount of ascorbic acid is about 1%, 1.5%, 2%, 2.5%, 3%, 3.3%, 3.5%, 3.8%, 4%, 4.5% or 5% by total weight of the lyophilized powder. In one embodiment, the amount of ascorbic acid is about 3.3% by total weight of the lyophilized powder.

In certain embodiments, the sodium bisulfite is present in an amount from about 5% to about 15% or about 8% to about 12% by total weight of the lyophilized powder. In certain embodiments, the sodium bisulfite is present in an amount from about 5%, 6%, 7%, 8%, 9%, 10%, 10.3%, 10.5%, 10.8%, 11%, 11.5%, 12% or 15% by total weight of the lyophilized powder. In one embodiment, the amount of sodium bisulfite is about 10.8% by total weight of the lyophilized powder.

In one embodiment, the antioxidant is a combination of ascorbic acid, sodium sulfite and sodium bisulfite. In one embodiment, the amount of ascorbic acid in the lyophilized powder is about 3.3%, the amount of sodium sulfite is about 3.3% and the amount of sodium bisulfite is about 10.8% by total weight of the lyophilized powder In one embodiment, the lyophilized powder also contains one or more of the following excipients: a buffer, such as sodium or potassium phosphate, or citrate; and a bulking agent, such as glucose, dextrose, maltose, sucrose, lactose, sorbitol, mannitol, glycine, polyvinylpyrrolidone, dextran. In one embodiment, the bulking agent is selected from dextrose, D-mannitol or sorbitol.

In certain embodiments, the lyophilized powders provided herein contain a phosphate buffer. In certain embodiments, the phosphate buffer is present in a concentration of about 10 mM, about 15 mM, about 20 mM, about 25 mM or about 30 mM. In certain embodiments, the phosphate buffer is present in a concentration of 20 mM. In certain embodiments, the phosphate buffer is present in a concentration of 20 mM, and the constituted formulation has a pH of about 7.

In certain embodiments, the lyophilized powders provided herein contain a citrate buffer. In one embodiment, the citrate buffer is sodium citrate dihydrate. In certain embodiments, the amount of sodium citrate dihydrate is from about 5% to about 15%, about 6% to about 12% or about 7% to about 10% by total weight of the lyophilized powder. In certain embodiments, the amount of sodium citrate dihydrate in the lyophilized powder is about 5%, 6%, 7%, 7.5%, 8%, 8.3%, 8.5%, 8.8%, 9%, 9.5%, 10%, 12% or about 15% by total weight of the lyophilized powder. In certain embodiments, the constituted formulation has a pH of about 5 to 10, or about 6.

In certain embodiments, the lyophilized powder provided herein contains dextrose in an amount ranging from about 30% to about 60% by total weight of the lyophilized powder. In certain embodiments, the amount of dextrose is about 30%, 35%, 40%, 45%, 50% or 60% by total weight of the lyophilized powder. In certain embodiments, the amount of dextrose is about 40% by total weight of the lyophilized powder. In certain embodiments, the lyophilized powder provided herein contains mannitol in an amount ranging from about 20% to about 50% by total weight of the lyophilized powder. In certain embodiments, the amount of mannitol is about 20%, 25%, 30%, 32%, 32.5%, 32.8%, 33%, 34%, 37%, 40%, 45% or 50% by total weight of the lyophilized powder. In certain embodiments, the amount of mannitol is about 32.8% by total weight of the lyophilized powder.

In certain embodiments, the lyophilized powder provided herein contains about 41% of sitaxsentan sodium, about 3.3% ascorbic acid, about 3.3% sodium sulfite and about 10.8% mg sodium bisulfite, about 8.8% sodium citrate dihydrate and about 32.8% mannitol by total weight of the lyophilized powder. In certain embodiments, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 53.5 |
| L-Ascorbic Acid | 20.0 |
| D-Mannitol | 200.0 |
| Sodium Bisulfite | 66.0 |
| Sodium Sulfite | 20.0 |
| Sodium Hydroxide or Hydrochloride Acid | QS to pH 6 |

In certain embodiments, the lyophilized powder provided herein contains about 40 to about 30% of sitaxsentan sodium, about 4 to about 6% ascorbic acid, about 6 to about 8% sodium citrate dihydrate, about 50 to about 60% D-mannitol and about 1 to about 2% citric acid monohydrate by total weight of the lyophilized powder. In certain embodiments, the lyophilized powder provided herein contains about 33% of sitaxsentan sodium, about 5.3% ascorbic acid, about 7.6% sodium citrate dihydrate, about 53% D-mannitol and 0.13% citric acid monohydrate by total weight of the lyophilized powder. In one embodiment, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 57.1 |
| L-Ascorbic Acid | 40.0 |
| D-Mannitol | 400.0 |
| Citric Acid Monohydrate | 1.3 |
| Sodium Hydroxide or Hydrochloride Acid | QS to pH 6.8 |

In certain embodiments, the lyophilized powder provided herein contains about 40 to about 30% of sitaxsentan sodium, about 4 to about 6% ascorbic acid, about 3 to about 4% sodium phosphate dibasic heptahydrate, about 50 to about 60% D-mannitol and about 1.5 to about 2.5% sodium phosphate monobasic monohydrate by total weight of the lyophilized powder. In certain embodiments, the lyophilized powder provided herein contains about 34% of sitaxsentan sodium, about 5.5% ascorbic acid, about 3.7% sodium phosphate dibasic heptahydrate, about 55% D-mannitol and 1.9% sodium phosphate monobasic monohydrate by total weight of the lyophilized powder. In one embodiment, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Phosphate Dibasic Heptahydrate | 26.8 |
| L-Ascorbic Acid | 40.0 |
| D-Mannitol | 400.0 |
| Sodium Phosphate Monobasic | 13.9 |
| Monohydrate | |
| Sodium Hydroxide or Hydrochloride Acid | QS to pH 6.8 |

The lyophilized formulations of sitaxsentan sodium provided herein can be administered to a patient in need thereof using standard therapeutic methods for delivering sitaxsentan sodium including, but not limited to, the methods described herein. In one embodiment, the lyophilized sitaxsentan sodium is administered by dissolving a therapeutically effective amount of the lyophilized sitaxsentan sodium provided herein in a pharmaceutically acceptable solvent to produce a pharmaceutically acceptable solution, and administering the solution (such as by intravenous injection) to the patient.

The lyophilized sitaxsentan sodium formulation provided herein can be constituted for parenteral administration to a patient using any pharmaceutically acceptable diluent. Such diluents include, but are not limited to Sterile Water for Injection, USP, Sterile Bacteriostatic Water for Injection, saline, USP (benzyl alcohol or parabens preserved). Any quantity of diluent may be used to constitute the lyophilized sitaxsentan sodium formulation such that a suitable solution for injection is prepared. Accordingly, the quantity of the diluent must be sufficient to dissolve the lyophilized sitaxsentan sodium. Typically, 10-50 mL or 10 to 20 mL of a diluent are used to constitute the lyophilized sitaxsentan sodium formulation to yield a final concentration of, about 1-50 mg/mL, about 5-40 mg/mL, about 10-30 mg/mL or 10-25 mg/mL. In certain embodiments, the final concentration of sitaxsentan sodium in the reconstituted solution is about 25 mg/mL or about 12.5 mg/mL. The precise amount depends upon the indication treated. Such amount can be empirically determined. In some embodiments, the pH of the reconstituted solution is about 5 to about 10 or about 6 to about 8. In some embodiments, the pH of the reconstituted solution is about 5, 6, 7, 8, 9 or 10.

Constituted solutions of lyophilized sitaxsentan sodium can be administered to a patient promptly upon constitution. Alternatively, constituted solutions can be stored and used within about 1-72 hours, about 1-48 hours or about 1-24 hours. In some embodiments, the solution is used within 1 hour of preparation.

e. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Sitaxsentan sodium may be formulated for local or topical application, such as for topical application to the skin and mucous membranes, in the form of gels, creams, and lotions. Topical administration is contemplated for transdermal delivery and also for administration mucosa, or for inhalation therapies.

f. Compositions for Other Routes of Administration

Other routes of administration, such as topical application, transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

g. Articles of Manufacture

The polymorph or mixture of polymorphs may be packaged as articles of manufacture containing packaging material and a label that indicates that sitaxsentan sodium is used for treating diastolic heart failure. The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of sitaxsentan provided herein are contemplated herein.

Dosages

In human therapeutics, the physician will determine the dosage regimen that is most appropriate according to a preventive or curative treatment and according to the age, weight, stage of the disease and other factors specific to the subject to be treated. In certain embodiments, dose rates of sitaxsentan sodium are from about 1 to about 350 mg per day for an adult, from about 1 to about 300 mg per day, from about 5 to about 250 mg per day, from about 5 to about 250 mg per day or from about 10 to 50 mg per day for an adult. Dose rates of from about 50 to about 300 mg per day are also contemplated herein. In certain embodiments, doses are about 5 mg, 10 mg, 15 mg, 20 mg, 25 mg, 30 mg, 35 mg, 40 mg, 45 mg, 50 mg, 60 mg, 70 mg, 80 mg, 100 mg, 125 mg, 150 mg, 175 mg or 200 mg per day per adult.

The amount of sitaxsentan sodium in the formulations provided herein which will be effective in the prevention or treatment of the diastolic heart failure or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 3 milligrams per kilogram, from about 10 micrograms per kilogram to about 3 milligrams per kilogram, from about 100 micrograms per kilogram to about 3 milligrams per kilogram, or from about 100 microgram per kilogram to about 2 milligrams per kilogram). In certain embodiments, the amount of sitaxsentan sodium administered is from about 0.01 to about 3 mg/kg for a subject in need thereof. In certain embodiments, the amount of sitaxsentan sodium administered is about 0.01, 0.05, 0.1, 0.2, 0.4, 0.8, 1.5, 2, 3 mg/kg of a subject. In the certain embodiments, the administration of sitaxsentan sodium is by intravenous injection.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

The amounts sufficient to prevent, manage, treat or ameliorate the symptoms of diastolic heart failure, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In another embodiment, the dosage of the formulation provided herein is administered to prevent, treat, manage, or ameliorate the symptoms of diastolic heart failure in a subject in a unit dose of from about 1 mg to 300 mg, 50 mg to 250 mg or 75 mg to 200 mg.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### F. Evaluation of the Activity

Standard physiological, pharmacological and biochemical procedures are available and are known to one of skill in the art to test the efficacy of sitaxsentan sodium in the methods provided herein. See, e.g., U.S. Pat. No. 5,114,918, EP 0 436 189 A1, Borges, et al. (1989) Eur. J. Pharm. 165: 223-230; Filip et al. (1991) Biochem. Biophys. Res. Commun. 177: 171-176. For example, efficacy evaluation of sitaxsentan sodium in the treatment of DHF can be conducted by routine tests including, but not limited to treadmill exercise test conducted at periodic intervals during the treatment; determining the effect on ventricular structure and function (i.e., left ventricular mass) according to echocardiography (ECHO); determining the ratio of transmittal inflow velocity (E) to early diastolic velocity of the mitral annulus (E′) according to Doppler ECHO and tissue Doppler imaging (TDI); determining the change in quality of life (QOL) measured by the Minnesota Living with Heart Failure questionnaire (MLHF); and Functional class assessments (NYHA). For example, see, Zile et al. Heart failure with a normal ejection fraction: is measurement of diastolic heart failure necessary to make the diagnosis of diastolic heart failure?, Circulation 2001; 104:779-782 and Miguel et al., Recommendations for quantification of Doppler echocardiography: a report from the Doppler quantification task force of the nomenclature and standards committee of the American society of echocardiography, J. Am. Soc. Echocardiogr. 2002; 15: 167-84.

### G. Combination Therapy

In the methods provided herein, the polymorph or mixture of polymorphs may, for example, be employed alone, in combination with one or more other endothelin antagonists, or with another compound or therapies useful for the treatment of diastolic heart failure. For example, the formulations can be administered in combination with other compounds known to modulate the activity of endothelin receptor, such as the compounds described in U.S. Pat. Nos. 6,432,994; 6,683,103; 6,686,382; 6,248,767; 6,852,745; 5,783,705; 5,962,490; 5,594,021; 5,571,821; 5,591,761; 5,514,691. Several other endothelin antagonists are described in the literature as described above.

In some embodiments, the methods involve administration of sitaxsentan sodium in combination with other compounds used in treatment of diastolic heart failure. Such agents include, but are not limited to loop diuretics such as Bumex® (bumetanide), Lasix® (furosemide), Demadex® (torsemide); thiazide diuretics such as Hygroton® (chlorthalidone), Hydrodiuril®, Esidrix® (HCTZ, hydrochlorothiazide), Amiloride, Aldactone® (spironolactone); long-acting nitrates, such as Isordil®, Sorbitrate® (Isosorbide Dinitrate), Imdur® (Isosorbide mononitrate); β-blockers such as bisoprolol fumarate, propranolol, atenolol, labetalol, sotalol, carvedilol; calcium channel blockers, such as Norvasc® (amlodipine), Cardizem® (diltiazem), Isoptin® (verapamil), Procardia® (nifedipine); renal artery stenosis (RAS) inhibitors and angiotensin converting enzyme (ACE) inhibitors, such as captopril, fosinopril, benazepril, enalapril, lisinopril, moexipril, perindopril, quinapril, ramipril, spirapril, trandolapril; angiotensin receptor blockers (ARBs), such as losartan, valsartan, irbesartan, telmesartan, and aldosterone antagonists.

In some embodiments, the methods involve administration of sitaxsentan sodium in combination with other compounds used in treatment of an interstitial disease, such as corticosteroids, for example, prednisone or methylprednisone, which are used to suppress active ongoing alveolar and interstitial inflammation and injury and in treating patients with interstitial lung disease.

Further, the polymorphs provided herein can be employed in combination with endothelin antagonists known in the art and include, but are not limited to a fermentation product of Streptomyces misakiensis, designated BE-18257B which is a cyclic pentapeptide, cyclo(D-Glu-L-Ala-allo-D-Ile-L-Leu-D-Trp); cyclic pentapeptides related to BE-18257B, such as cyclo(D-Asp-Pro-D-Val-Leu-D-Trp) (BQ-123) (see, U.S. Pat. No. 5,114,918 to Ishikawa et al.; see, also, EP A10 436 189 to BANYU PHARMACEUTICAL CO., LTD (Oct. 7, 1991)); and other peptide and non-peptidic ETA antagonists have been identified in, for example, U.S. Pat. Nos. 6,432,994; 6,683,103; 6,686,382; 6,248,767; 6,852,745; 5,783,705; 5,962,490; 5,594,021; 5,571,821; 5,591,761; 5,514,691; 5,352,800; 5,334,598; 5,352,659; 5,248,807; 5,240,910; 5,198,548; 5,187,195; 5,082,838; 6,953,780; 6,946,481; 6,852,745; 6,835,741; 6,673,824; 6,670,367; and 6,670,362. These include other cyclic pentapeptides, acyltripeptides, hexapeptide analogs, certain anthraquinone derivatives, indanecarboxylic acids, certain N-pyriminylbenzenesulfonamides, certain benzenesulfonamides, and certain naphthalenesulfonamides (Nakajima et al. (1991) J. Antibiot. 44:1348-1356; Miyata et al. (1992) J. Antibiot. 45:74-8; Ishikawa et al. (1992) J. Med. Chem. 35:2139-2142; U.S. Pat. No. 5,114,918 to Ishikawa et al.; EP A1 0 569 193; EP A1 0 558 258; EP A1 0 436 189 to BANYU PHARMACEUTICAL CO., LTD (Oct. 7, 1991); Canadian Patent Application 2,067,288; Canadian Patent Application 2,071,193; U.S. Pat. No. 5,208,243; U.S. Pat. No. 5,270,313; U.S. Pat. No. 5,612,359, U.S. Pat. No. 5,514,696, U.S. Pat. No. 5,378,715; Cody et al. (1993) Med. Chem. Res. 3:154-162; Miyata et al. (1992) J. Antibiot 45:1041-1046; Miyata et al. (1992) J. Antibiot 45:1029-1040, Fujimoto et al. (1992) FEBS Lett. 305:41-44; Oshashi et al. (1002) J. Antibiot 45:1684-1685; EP A1 0 496 452; Clozel et al. (1993) Nature 365:759-761; International Patent Application WO93/08799; Nishikibe et al. (1993) Life Sci. 52:717-724; and Benigni et al. (1993) Kidney Int. 44:440-444). Numerous sulfonamides that are endothelin peptide antagonists are also described in U.S. Pat. Nos. 5,464,853; 5,594,021; 5,591,761; 5,571,821; 5,514,691; 5,464,853; International PCT application No. 96/31492; and International PCT application No. WO 97/27979.

Further endothelin antagonists described in the following documents, incorporated herein by reference in their entirety, are exemplary of those contemplated for use in combination with the polymorphs provided herein: U.S. Pat. No. 5,420,123; U.S. Pat. No. 5,965,732; U.S. Pat. No. 6,080,774; U.S. Pat. No. 5,780,473; U.S. Pat. No. 5,543,521; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016; GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ETA Receptor”, Life Sciences, Vol. 50(4), pp. 247-255 (1992)); PD 156707 (Reynolds, E., et al., “Pharmacological Characterization of PD 156707, an Orally Active ETA Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”, The Journal of Pharmacology and Experimental Therapeutics, Vol. 275(3), pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91, pp. 8052-8056 (1994)); SB 217242 (Ohlstein, E. H., et al., “Nonpeptide Endothelin Receptor Antagonists. VI: Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-127722 (Opgenorth, T. J., et al., “Pharmacological Characterization of A-127722: An Orally Active and Highly Potent E.sub.TA-Selective Receptor Antagonist”, The Journal of Pharmacology and Experimental Therapeutics, Vol. 276(2), pp. 473-481 (1996)); TAK-044 (Masuda, Y., et al., “Receptor Binding and Antagonist Properties of a Novel Endothelin Receptor Antagonist, TAK-044 {Cyclo [D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human EndothelinA and EndothelinB Receptors”, The Journal of Pharmacology and Experimental Therapeutics, Vol. 279(2), pp. 675-685 (1996)); bosentan (Ro 47-0203, Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)).

The polymorphs provided herein can also be administered in combination with other classes of compounds. Exemplary classes of compounds for combinations herein include endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abdximab, eptifibatide, and tirofiban). P2Y(AC) antagonists (e.g., clopidogrel, ticlopidine and CS-747), and aspirin; anticoagulants such as warfarin, low molecular weight heparins such as enoxaparin, Factor VIIa Inhibitors, and Factor Xa Inhibitors, renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase Inhibitors such as pravastatin, lovastatin, atorvastatin, simvastatin, NK-104 (a.k.a. itavastatin, or nisvastatin or nisbastatin) and ZD-4522 (also known as rosuvastatin, or atavastatin or visastatin); squalene synthetase inhibitors; fibrates; bile acid sequestrants such as questran; niacin; anti-atherosclerotic agents such as ACAT inhibitors; MTP Inhibitors: calcium channel blockers such as amlodipine besylate; potassium channel activators; alpha-adrenergic agents, beta-adrenergic agents such as carvedilol and metoprolol; antiarrhythmic agents; diuretics, such as chlorothlazide, hydrochlorothiazide, flumethiazide, hydroflumethiazide, bendroflumethiazide, methylchlorothiazide, trichloromethiazide, polythiazide or benzothlazide as well as ethacrynic acid, tricrynafen, chlorthalidone, furosenilde, musolimine, bumetanide, triamterene, amiloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists such as spironolactone and eplerenone; growth hormone secretagogues; aP2 inhibitors; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil, tadalafil, vardenafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes): antimetabolites such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives: microtubule-stablizing agents such as pacitaxel (Taxol®), docetaxel (Taxotere®), and epothilones A-F or their analogs or derivatives; plant-derived products, such as vinca alkaloids, epipodophyllotoxins, taxanes; and topoisomerase inhibitors: prenyl-protein transferase inhibitors: and miscellaneous agents such as, hydroxyurea, procarbazine, mitotane, hexamethylmelamine, platinum coordination complexes such as cisplatin, satraplatin, and carboplatin); cyclosporins; steroids such as prednisone or dexamethasone; gold compounds; cytotoxic drugs such as azathiprine and cyclophosphamide: TNF-alpha inhibitors such as tenidap; anti-TNF antibodies or soluble TNF receptor such as etanercept (Enbrel) rapamycin (sirolimus or Rapamune), leflunimide (Arava); and cyclooxygenase-2 (COX-2) inhibitors such as celecoxib (Celebrex) and rofecoxib (Vioxx).

The above other therapeutic agents may be used, for example, in those amounts indicated in the Physicians' Desk Reference (PDR) or as otherwise determined by one of ordinary skill in the art.

### H. Methods of use of the Polymorphs of N-(4-chloro-3-methyl-5-isoxazolyl)-2-[2-methyl-4,5-(methylenedioxy)phenyl-acetyl]thiophene-3-sulfonamide, Sodium Salt

N-(4-chloro-3-methyl-5-isoxazolyl)-2-[2-methyl-4,5-(methylenedioxy)phenyl-acetyl]thiophene-3-sulfonamide, sodium salt in polymorph Forms A, B and C are useful in the treatment of endothelin-mediated diseases. These treatments encompass administering to a subject an effective amount of Forms A, B or C, wherein the effective amount is sufficient to ameliorate one or more of the symptoms of the disease.

Polymorph A, B and C are effective for the treatment of hypertension, cardiovascular diseases, cardiac diseases including myocardial infarction, pulmonary hypertension, neonatal pulmonary hypertension, erythropoietin-mediated hypertension, respiratory diseases and inflammatory diseases, including asthma, bronchoconstriction, opthalmologic diseases including glaucoma and inadequate retinal perfusion, gastroenteric diseases, renal failure, endotoxin shock, menstrual disorders, obstetric conditions, wounds, laminitis, erectile dysfunction, menopause, osteoporosis and metabolic bone disorders, climacteric disorders including hot flashes, abnormal clotting patterns, urogenital discomfort and increased incidence of cardiovascular disease and other disorders associated with the reduction in ovarian function in middle-aged women, pre-eclampsia, control and management of labor during pregnancy, nitric oxide attenuated disorders, anaphylactic shock, hemorrhagic shock, interstitial lung disease, diastolic heart failure and immunosuppressant-mediated renal vasoconstriction. In one embodiment, the disease is pulmonary hypertension.

Polymorphs A, B and C are also useful for inhibiting the binding of an endothelin peptide to an endothelin_{A} (ET_{A}) or endothelin_{B} (ET_{B}) receptor. This inhibiting encompasses contacting the receptor with any of the polymorphs A, B or C, or o pharmaceutially acceptable derivative thereof, wherein the contacting is effected prior to, simultaneously with or subsequent to contacting the receptor with the endothelin peptide.

Polymorphs A, B and C are also useful for altering endothelin receptor-mediated activity. This altering encompasses contacting an endothelin receptor with any of the polymorphs A, B or C.

The following examples are included for illustrative purposes only and are not intended to limit the scope of the claimed subject matter. N-(4-chloro-3-methyl-5-isoxazolyl)-2-[2-methyl-4,5-(methylenedioxy)phenylacetyl]thiophene-3-sulfonamide, sodium salt (sitaxsentan sodium) may be prepared by the procedure described in International (PCT) Patent Application Publication No. WO 98/49162.

### EXAMPLE 1

### Production Procedure with MeOH

10 g Sitaxsentan sodium was suspended in 40 mL iPrOAc, 30 mL EtOH and 30 mL MeOH and heated at 75° C. until a clear solution was obtained. The solution was allowed by cool to RT and the solution remained clear for 1 hour. 50 mL MTBE was added and over time light yellow solids were formed. The solids were collected via filtration, washed with MTBE and dried under vacuum to yield 6.4 g Sitaxsentan sodium as mostly polymorph A.

### EXAMPLE 2

### Production procedure without MeOH

10 g Sitaxsentan sodium was suspended in 100 mL iPrOAc and 80 mL EtOH and heated to 90° C. (reflux) until a clear solution was obtained (the amount of solvents were necessary to obtain a clear solution at reflux). The solution was allowed by cool to RT and the solution remained clear for 1 hour. 140 mL MTBE was added and a very small amount of light yellow solids were formed over time (addition of less MTBE did not result in the formation of solids). 100 mL MTBE was added and more light yellow solids were formed over time. These solids were obtained by decanting the solution (these solids to not filter well), washed with MTBE and dried under vacuum to yield 5.4 g Sitaxsentan sodium as mostly polymorph B.

### EXAMPLE 3

### Recystallization from Wet iPrOH

10 g Sitaxsentan sodium was suspended in 100 mL iPrOH and 5 mL water and heated to 100° C. (reflux) until a clear solution was obtained. This solution was allowed to cool to RT upon which light yellow solids were formed. These solids were collected via filtration, washed with iPrOH and dried under vacuum to yield 6.4 g Sitaxsentan sodium lot as mostly polymorph A.

### EXAMPLE 4

### Recrystallization of Sitaxsentan Sodium Polymorph B

1.0 g Sitaxsentan sodium, from Example 5 was suspended in 1.6 mL iPrOAc, 1.6 mL MeOH and 1.6 mL EtOH and placed in an oil bath preheated at 65° C. Complete dissolution was obtained in 5 minutes. The solution was allowed to cool to room temperature and the solution remained clear. Upon standing for days a light yellow solids was formed which was collected via filtration, washed with MTBE and dried under vacuum to yield Sitaxsentan sodium as ˜94% polymorph A.

### EXAMPLE 5

### Recrystallization of Sitaxsentan Sodium Polymorph A

688 g of Sitaxsentan sodium was heated for 30 min at 60° C. in 7.8 L EtOH, then cooled to 10° C. MTBE (35 L) was added and the mixture was filtered at 10° C. The solid was dried in vacuo to provide approximately 86:14 polymorph A:polymorph B.

### EXAMPLE 6

387 g of Sitaxsentan sodium was slurried in 3.0 L of isopropanol for 2 h, then cooled for 2 days at 5° C. The solid was filtered and dried in vacuo to give 344 g of Sitaxsentan sodium. This material was slurried in 1.72 L of isopropanol at r.t. for 30 min, then cooled to 5° C. for 45 min. The product was filtered and dried in vacuo to provide mostly polymorph B.

### EXAMPLE 7

Sitaxsentan sodium (23.4 kg) was suspended in isopropyl acetate (32.8 kg), ethanol (30 kg), and methanol (30 kg) and heated to 65° C. After the solids had dissolved, the solution was hot filtered through a 0.45 micron filter. The filtrate was agitated and cooled to 45° C. Seed crystals of sitaxsentan sodium were added and agitation of the contents was continued at a temperature of 45° C. for 3 hours. MTBE (164.3 kg) was slowly added at 45±5° C. and at a rate exceeding 1 kg per minute. The contents were slowly cooled to 0° C. over a period of 4.5 hours. Agitation was continued at 0° C. for an additional 4.5 hours. The crystal crop was filtered and the wet cake washed with MTBE (93.6 kg). The wet cake was held under nitrogen until de-liquored. The wet cake was dried with gentle agitation at 40° C. in a Filter/Dryer until the level of residual MTBE was less than 500 ppm. The resulting material was mostly polymorph A (95:5±3 polymorph A:polymorph B).

### EXAMPLE 8

### Comparative Example

### Formation of Sitaxsentan Sodium:

To a well stirred suspension of 10 g Sitaxsentan sodium in 50 mL DCM was added 50 mL 2 N HCl followed by addition of MeOH until a clear solution was obtained. The two layers were separated and the organic layer was dried over MgSO₄ and concentrated to complete dryness in vacuo to give Sitaxsentan (˜9 g) as dry yellow foam.

### A. First Crystallization:

Sitaxsentan was redissolved in 100 mL EtOAc and washed with 3×50 mL saturated NaHCO3, brine, dried over MgSO4 and concentrated to dryness in vacuo. This material was resuspended in DCM to form a hazy solution and stirred for 5 minutes after which light yellow solids were formed. 150 mL Et2O was added. The solids were collected via filtration, washed with 1 to 2 DCM to Et2O and dried under vacuum to yield Sitaxsentan sodium as mostly amorphous material.

### B. Second Crystallization:

Sitaxsentan sodium was redissolved in 200 mL water and acidified to pH ˜2 with conc. HCl upon which a very light yellow solid was formed. This solid was obtained via filtration. This material was redissolved in 100 mL EtOAc and washed with 50 mL brine, 2×50 mL saturated NaHCO3, brine, dried over MgSO4 and concentrated to dryness in vacuo. This material was resuspended in DCM to form a hazy solution and stirred for 5 minutes after which light yellow solids were formed. 150 mL Et2O was added. The solids were collected via filtration, washed with 1 to 2 DCM to Et2O and dried under vacuum to yield 6.1 g Sitaxsentan sodium as mostly amorphous material.

### C. Third Crystallization:

1.0 Sitaxsentan sodium was suspended in 10 mL EtOH and heated to reflux until a clear solution was obtained (the amount of solvent was necessary to obtain a clear solution). This solution was allowed to cool to RT and remained clear for 1 hour. At this point 15 mL MTBE was added and the solution turns turbid (addition of 10 mL MTBE did not result in the formation of solids within 30 minutes). This solution was heated to reflux but this did not prevent crashing out of solids. The solids were collected via filtration, washed with MTBE and dried under vacuum to yield 0.64 g Sitaxsentan sodium as a mix of amorphous and crystalline material.

Since modifications will be apparent to those of skill in this art, it is intended that the claimed subject matter be limited only by the scope of the appended claims.

What is claimed is:

1. A compound N-(4-chloro-3-methyl-5-isoxazolyl)-2-[2-methyl-4,5-(methylenedioxy)phenylacetyl]thiophene-3-sulfonamide, sodium salt, in a form of polymorph A.

2. The compound of claim 1, wherein an amount of polymorph A is more than about 80%.

3. The compound of claim 1, wherein the amount of polymorph A is more than about 85%.

4. The compound of claim 1, wherein the amount of polymorph A is more than about 90%.

5. The compound of claim 1, wherein the amount of polymorph A is more than about 95%.

6. The compound of claim 1, wherein the amount of polymorph A is more than about 98%.

7. The compound of claim 1, wherein the amount of polymorph A is more than about 99%.

8. The compound of claim 1, wherein the amount of polymorph A is about 100%.

9. A compound N-(4-chloro-3-methyl-5-isoxazolyl)-2-[2-methyl-4,5-(methylenedioxy)phenylacetyl]thiophene-3-sulfonamide, sodium salt, in a form of a mixture of polymorphs A and B, wherein a ratio of polymorph A:B is greater than or is equal to about 80:20.

10. The compound of claim 9, wherein the ratio of A:B is greater than or is equal to about 86:14.

11. The compound of claim 9, wherein the ratio of A:B is greater than or is equal to about 90:10.

12. The compound of claim 9, wherein the ratio of A:B is greater than or is equal to about 91:9.

13. The compound of claim 9, wherein the ratio of A:B is greater than or is equal to about 92:8.

14. The compound of claim 9, wherein the ratio of A:B is greater than or is equal to about 93:7.

15. The compound of claim 9, wherein the ratio of A:B is greater than or is equal to about 94:6.

16. The compound of claim 9, wherein the ratio of A:B is greater than or is equal to about 95:5.

17. The compound of claim 9, wherein the ratio of A:B is greater than or is equal to about 96:4.

18. The compound of claim 9, wherein the ratio of A:B is greater than or is equal to about 97:3.

19. The compound of claim 9, wherein the ratio of A:B is greater than or is equal to about 98:2.

20. The compound of claim 9, wherein the ratio of A:B is greater than or is equal to about 99:1.

21. The compound of claim 1, wherein the polymorph A is characterized by peaks in the XRPD pattern at approximately 22.38 and 23.38 degrees 2-theta.

22. The compound of claim 1, wherein the polymorph A is characterized by peaks in the XRPD pattern at approximately 6.72, 15.96, 22.38, 23.38 and 26.22 degrees 2-theta.

23. The compound of claim 1, wherein the polymorph A is characterized by a peak in the Raman spectra at approximately 1602.1 cm⁻¹.

24. The compound of claim 1, wherein the polymorph A is characterized by peaks in the Raman spectra at approximately 1697.4, 1602.1, 1489.8 and 1402.2 cm⁻¹.

25. A process for producing the polymorph A as defined in claim 1, comprising the steps of:
dissolving N-(4-chloro-3-methyl-5-isoxazolyl)-2-[2-methyl-4,5-(methylenedioxy)phenylacetyl]thiophene-3-sulfonamide, sodium in a warm solvent to afford a saturated solution; and
cooling the saturated solution to obtain a solid precipitate.

26. The process of claim 25, wherein the solvent is acetonitrile, chloroform, dichloromethane, ethanol, ethyl acetate, hexanes, isopropanol, isopropyl acetate, methyl t-butyl ether methyl ethyl ketone, toluene or tetrahydrofuran.

27. The process of claim 25, wherein the solvent is ethanol and the saturated solution is slowly cooled to an ambient temperature.

28. The process of claim 25, wherein the saturated solution is a slurry.

29. The process of claim 25, wherein the solvent is ethanol and the solid precipitate is filtered within one or more hours after it has precipitated.

30. A process for producing the polymorph A as defined in claim 1, comprising the steps of:
dissolving N-(4-chloro-3-methyl-5-isoxazolyl)-2-[2-methyl-4,5-(methylenedioxy)phenylacetyl]thiophene-3-sulfonamide, sodium in a solvent to afford a saturated solution; and
adding an antisolvent.

31. The process of claim 30, wherein the solvent is tetrahydrofuran and the antisolvent is hexanes.

32. The process of claim 30, wherein the solvent is methanol and the antisolvent is toluene.

33. The process of claim 30, wherein the solvent comprises isopropyl acetate, ethanol and methanol.

34. The process of claim 33, further comprising a step of heating the solvent up to about 65° C.

35. The process of claim 33, wherein the antisolvent is methyl t-butyl ether.

36. The process of claim 35, wherein the methyl t-butyl ether is added at a temperature of about 45±5° C.

37. The process of claim 36, further comprising a step of cooling up to about 0° C.

38. The process of claim 37, wherein the cooling step is carried out over a period of about 3.5 to 4.5 hours.

39. A compound N-(4-chloro-3-methyl-5-isoxazolyl)-2-[2-methyl-4,5-(methylenedioxy)phenylacetyl]thiophene-3-sulfonamide, sodium salt, in a form of polymorph B, wherein an amount of polymorph B in the compound is greater than about 70%.

40. The compound of claim 39, wherein the amount of polymorph B is more than about 80%.

41. The compound of claim 39, wherein the amount of polymorph B is more than about 85%.

42. The compound of claim 39, wherein the amount of polymorph B is more than about 90%.

43. The compound of claim 39, wherein the amount of polymorph B is more than about 95%.

44. The compound of claim 39, wherein the amount of polymorph B is more than about 98%.

45. The compound of claim 39, wherein the amount of polymorph B is more than about 99%.

46. The compound of claim 39, wherein the amount of polymorph B is about 100%.

47. The compound of claim 39, wherein the polymorph B is characterized by a peak in the XRPD pattern at approximately 22.72 degrees 2-theta.

48. The compound of claim 39, wherein the polymorph B is characterized by peaks in the XRPD pattern at approximately 6.6, 15.52, 18.38, 18.94 and 22.72 degrees 2-theta.

49. The compound of claim 39, wherein the polymorph B is characterized by a peak in the Raman spectra at approximately 1594.7 cm⁻¹.

50. The compound of claim 39, wherein the polymorph B is characterized by peaks in the Raman spectra at approximately 1696.9, 1594.7, 1490.2 and 1397.8 cm⁻¹.

51. A process for producing Form B as defined in claim 39, comprising the steps of:
dissolving N-(4-chloro-3-methyl-5-isoxazolyl)-2-[2-methyl-4,5-(methylenedioxy)phenylacetyl]thiophene-3-sulfonamide, sodium in a solvent to afford a saturated solution; and
adding an antisolvent to obtain a solid precipitate.

52. The process of claim 51, wherein the solvent is ethyl acetate and the antisolvent is hexanes, methyl t-butyl ether or toluene.

53. The process of claim 51, wherein the solvent is acetone and the antisolvent is dichloromethane, methyl t-butyl ether or toluene.

54. The process of claim 51, wherein the solvent is tetrahydrofuran and the antisolvent is methyl t-butyl ether.

55. The process of claim 51, wherein the solvent is isopropyl acetate and the antisolvent is methyl t-butyl ether.

56. The process of claim 51, wherein the solvent is ethanol and the antisolvent is methyl t-butyl ether.

57. The process of claim 51, wherein the solvent is methanol and the antisolvent is methyl t-butyl ether.

58. The process of claim 51, wherein the antisolvent is added at a temperature of about 20° C.

59. The process of claim 58, further comprising a step of cooling.

60. The process of claim 59, wherein the cooling is carried over a period of about 3 hours up to 0° C.

61. The process of claim 60, wherein the solid precipitate is filtered within one or more hours after it has precipitated.

62. A method for the treatment of an endothelin-mediated disease, comprising administering to a subject an effective amount of the polymorph of claim 1.

63. The method of claim 62, wherein the disease is selected from a group consisting of hypertension, cardiovascular disease, cardiac disease, pulmonary hypertension, neonatal pulmonary hypertension, erythropoietin-mediated hypertension, respiratory disease, inflammatory disease, opthalmologic disease, gastroenteric disease, renal failure, endotoxin shock, menstrual disorder, obstetric condition, wound, laminitis, erectile dysfunction, menopause, osteoporosis, metabolic bone disorder, climacteric disorder, disorder associated with the reduction in ovarian function in middle-aged women, pre-eclampsia, management of labor during pregnancy, nitric oxide attenuated disorder, anaphylactic shock, interstitial lung disease, diastolic heart failure, hemorrhagic shock and immunosuppressant-mediated renal vasoconstriction.

65. The method of claim 62, wherein the disease is pulmonary hypertension.

66. A method for inhibiting the binding of an endothelin peptide to an endothelin_{A} (ET_{A}) or endothelin_{B} (ET_{B}) receptor, comprising contacting the receptor with the polymorph of claim 1, wherein:
the contacting is effected prior to, simultaneously with or subsequent to contacting the receptor with the endothelin peptide.

67. A method for altering endothelin receptor-mediated activity, comprising contacting an endothelin receptor with the polymorph of claim 1.

68. A method for the treatment of an endothelin-mediated disease, comprising administering to a subject an effective amount of the polymorph of claim 9.

69. A method for inhibiting the binding of an endothelin peptide to an endothelin_{A} (ET_{A}) or endothelin_{B} (ET_{B}) receptor, comprising contacting the receptor with the polymorph of claim 9, wherein:
the contacting is effected prior to, simultaneously with or subsequent to contacting the receptor with the endothelin peptide.

70. A method for altering endothelin receptor-mediated activity, comprising contacting an endothelin receptor with the polymorph of claim 9.

71. A method for the treatment of an endothelin-mediated disease, comprising administering to a subject an effective amount of the polymorph of claim 39.

72. A method for inhibiting the binding of an endothelin peptide to an endothelin_{A} (ET_{A}) or endothelin_{B} (ET_{B}) receptor, comprising contacting the receptor with the polymorph of claim 39, wherein:
the contacting is effected prior to, simultaneously with or subsequent to contacting the receptor with the endothelin peptide.

73. A method for altering endothelin receptor-mediated activity, comprising contacting an endothelin receptor with the polymorph of claim 39.

74. A pharmaceutical composition, comprising the polymorph of claim 1 and a pharmaceutically acceptable carrier.

75. A pharmaceutical composition comprising the polymorph of claim 9 and a pharmaceutically acceptable carrier.

76. A pharmaceutical composition comprising the polymorph of claim 39 and a pharmaceutically acceptable carrier.

77. The pharmaceutical composition of claim 74, wherein the polymorph A is present in an amount that is about more than 70% of the total weight of the N-(4-chloro-3-methyl-5-isoxazolyl)-2-[2-methyl-4,5-(methylenedioxy)phenylacetyl]thiophene-3-sulfonamide sodium in the composition.

78. The pharmaceutical composition of claim 74, wherein the polymorph A is present in an amount that is about more than 80% of a total weight of the N-(4-chloro-3-methyl-5-isoxazolyl)-2-[2-methyl-4,5-(methylenedioxy)phenylacetyl]thiophene-3-sulfonamide sodium in the composition.

79. The pharmaceutical composition of claim 74, wherein the polymorph A is present in an amount that is about more than 85% of a total weight of the N-(4-chloro-3-methyl-5-isoxazolyl)-2-[2-methyl-4,5-(methylenedioxy)phenyl-acetyl]thiophene-3-sulfonamide sodium in the composition.

80. The pharmaceutical composition of claim 74, wherein the polymorph A is present in an amount that is about more than 90% of a total weight of the N-(4-chloro-3-methyl-5-isoxazolyl)-2-[2-methyl-4,5-(methylenedioxy)phenyl-acetyl]thiophene-3-sulfonamide sodium in the composition.

81. The pharmaceutical composition of claim 74, wherein the polymorph A is present in an amount that is about more than 95% of a total weight of the N-(4-chloro-3-methyl-5-isoxazolyl)-2-[2-methyl-4,5-(methylenedioxy)phenyl-acetyl]thiophene-3-sulfonamide sodium in the composition.

82. The pharmaceutical composition of claim 74, wherein the polymorph A is present in an amount that is about more than 99% of a total weight of the N-(4-chloro-3-methyl-5-isoxazolyl)-2-[2-methyl-4,5-(methylenedioxy)phenyl-acetyl]thiophene-3-sulfonamide sodium in the composition.

83. The pharmaceutical composition of claim 74, wherein the polymorph A is present in an amount that is about more than 99.5% of a total weight of the N-(4-chloro-3-methyl-5-isoxazolyl)-2-[2-methyl-4,5-(methylenedioxy)phenyl-acetyl]thiophene-3-sulfonamide sodium in the composition.

84. The pharmaceutical composition of claim 74, wherein the polymorph A is present in an amount that is about more than 99.9% of a total weight of the N-(4-chloro-3-methyl-5-isoxazolyl)-2-[2-methyl-4,5-(methylenedioxy)phenyl-acetyl]thiophene-3-sulfonamide sodium in the composition.

85. The pharmaceutical composition of claim 74, wherein the polymorph A is present in an amount that is about 100% of a total weight of the N-(4-chloro-3-methyl-5-isoxazolyl)-2-[2-methyl-4,5-(methylenedioxy)phenylacetyl] thiophene-3-sulfonamide sodium in the composition.

86. The composition of claim 74 that is formulated for single or multiple dosage administration.

87. The pharmaceutical composition of claim 74 that is formulated as an oral tablet.

88. The oral tablet of claim 87 further comprising an antioxidant, a binding agent, a diluent, a buffer and a moisture resistant coating.

89. The pharmaceutical composition of claim 75 that is formulated as an oral tablet.

90. The pharmaceutical composition of claim 76 that is formulated as an oral tablet.

91. A lyophilized powder comprising the polymorph of claim 1.

92. The lyophilized powder of claim 91 further comprising an antioxidant, a buffer and a bulking agent.

93. A lyophilized powder comprising the polymorph of claim 9.

94. A lyophilized powder comprising the polymorph of claim 39.

95. An article of manufacture, comprising packaging material and the polymorph of claim 1, contained within the packaging material, wherein the polymorph is effective for antagonizing the effects of endothelin, ameliorating the symptoms of an endothelin-mediated disorder, or inhibiting the binding of an endothelin peptide to an ET receptor and the packaging material includes a label that indicates that the polymorph is used for antagonizing the effects of endothelin, inhibiting the binding of endothelin to an endothelin receptor or treating an endothelin mediated disorder.

96. An article of manufacture, comprising packaging material and the polymorph of claim 9, contained within the packaging material, wherein the polymorph is effective for antagonizing the effects of endothelin, ameliorating the symptoms of an endothelin-mediated disorder, or inhibiting the binding of an endothelin peptide to an ET receptor and the packaging material includes a label that indicates that the polymorph is used for antagonizing the effects of endothelin, inhibiting the binding of endothelin to an endothelin receptor or treating an endothelin mediated disorder.

97. An article of manufacture, comprising packaging material and the polymorph of claim 39, contained within the packaging material, wherein the polymorph is effective for antagonizing the effects of endothelin, ameliorating the symptoms of an endothelin-mediated disorder, or inhibiting the binding of an endothelin peptide to an ET receptor and the packaging material includes a label that indicates that the polymorph is used for antagonizing the effects of endothelin, inhibiting the binding of endothelin to an endothelin receptor or treating an endothelin mediated disorder.
